(19) United States
(12) Patent Application Publication
(10) Pub. No.: US 2021/0210199 A1
(43) Pub. Date: Jul. 8, 2021

(54) HEALTHCARE PRACTICE MANAGEMENT SYSTEM AND METHOD THEREOF

(71) Applicant: Vericle Corporation, Clearwater, FL (US)

(72) Inventors: Yuval LIROV, Clearwater Beach, FL (US); Erez LIROV, Morganville, NJ (US)

(21) Appl. No.: 17/208,681

(22) Filed: Mar. 22, 2021

Related U.S. Application Data
(63) Continuation-in-part of application No. 16/248,725, filed on Jan. 15, 2019, now abandoned.

(51) Int. Cl. G16H 40/20; G16H 10/60; G06Q 10/10; G06Q 40/08; G16H 50/70; G16H 70/20; G06N 20/00

(52) U.S. Cl. CPC G16H 40/20; G16H 10/60; G06Q 10/10; G06N 20/00; G16H 50/70; G16H 70/20; G06Q 40/08

(57) ABSTRACT

Disclosed within are systems and methods that minimize float owed to healthcare service providers by healthcare insurance companies. The systems and methods described herein reduce overall float by creating a task list related to submitted or to-be submitted insurance claims so that problematic claims can be addressed and heuristically determined tasks generated that maximize the opportunity to obtain payment for the healthcare services provided and overall float in the system is minimized and managed.

### CROSS REFERENCE TO RELATED APPLICATIONS

This application is a continuation-in-part (CIP) of U.S. Ser. No. 16/248,725 filed Jan. 15, 2019 entitled HEALTHCARE PRACTICE MANAGEMENT SYSTEM AND METHOD THEREOF, the contents of which are incorporated herein by reference in its entirety.

### FIELD OF THE INVENTION

The embodiments described herein relate generally to a practice management framework for healthcare practice management, and more specifically to systems, methods, and modes for non-transitory subject matter that reduces or eliminates Payor float in a computer-implemented health insurance billing platform and associated software.

### BACKGROUND OF THE INVENTION

Healthcare institutions struggle to receive payments for their services from insurance companies and Payors of medical services. There is a constant battle between healthcare service providers (“Payees”) and insurance companies, such as Blue Cross Blue Shield and governmental organizations, such as Medicare and Medicaid, (“Payors”) to process claims and transmit payment for services provided.

Payees work diligently process claims so as to timely collect payment from Payors. Delays, however, are often present in transmitting and collecting insurance premiums from Payors that transmit payment. Payors, such as insurance companies, do not pay out all the money they collect right away. Rather, an insurance company will collect money in the form of premiums from its clients, invest that money, and then pay out claims as needed at some future date. The difference between premiums collected and claims paid out is insurance float or float. Float is, in essence, a loan to the Payors, or debt owed by the Payors to the Payees. System float (or total system float) refers to an entire amount of float (or money) owed to a single Payee, by one or more Payors.

In practice, healthcare institutions have large numbers of clients and submit thousands of claims to various Payors. Tracking the claims and payments and having payments processed is extraordinarily complex, as insurance companies have a built-in incentives not to pay legally valid claims under their contractual obligations and to maximize float, as it gives maximum amounts of time to invest money from clients. Furthermore, insurance companies intentionally fail to make payments or make underpayments in order to increase float and have additional time in which to invest float funds. Insurance companies also make the rules for submitting claims intentionally complex and dispute claims for the most trivial and minor of reasons. In short, it is the Payor's financial best interest to not timely pay any claim. Furthermore, it is to be noted that all or substantially all Payors require electronic communications, i.e., all claims must be submitted electronically. It is often the case that substantially all or all of the information regarding service provided by a healthcare service provider is entered and maintained electronically. Most patients are given a tablet when first visiting a healthcare service provider the first time to enter all their relevant contact, insurance, and medical history information to be processed by the healthcare service provider. The doctor(s) or nurse(s) or physician's assistants that provide the care also enter and store treatment information electronically. Even x-ray images are taken and stored in electronic format. All of this electronic processing is done to minimize human interaction with the data and to more efficiently process the treatment, record keeping, and submissions of claims to Payors.

For healthcare institutions, there is an incentive to minimize insurance float. However, tackling this task is difficult as it is often difficult to determine which insurance claims to target and to follow up on to have paid. The total number of decision possibilities in a typical healthcare institution practice is comparable to the number of possibilities in a chess game. A typical healthcare practice owner and/or practice management staff do not have the mental wherewithal to manage such complexities daily, and it is often impossible to choose the proper claim to work on during the day in order to have it paid so to reduce overall Payor float owed to the healthcare institution.

For example, a healthcare institution might have a patient roster of 500 patients. Each patient has their own Payor which has its own set of rules and complicated processes. In this example, complexity exists as processing the order of claims is often extremely laborious and time consuming.

For a larger practice, say 10 offices each containing 500 patients; complexity reaches thousands of decision points and it is often impossible to target the claims to focus on to reduce Payor float; as workflow reaches astronomical hyper-complexity.

Ideally, staff in healthcare institutions need to choose their best actions and perform each quickly, accurately, timely, and pressing the envelope of their own limitations and to target the claims to reduce Payor float. Given the astronomical number of potential complex choices, it is mentally impossible to ensure an optimal decision for each one.

This growing complexity negatively affects a practice in multiple ways. It exacerbates practice management challenges by inviting more errors which domino into errors causing more delays which adds to overall complexity and thus creating a vicious downward productivity cycle.

As staff make more mistakes in processing claims, often Payor float increases as Payor float delay payment in order to maximize profits.

Current methods and systems are not designed with the goal to minimize Payor float and with the aspects of complexity and Payor adversity in mind. As a result, practice owners and managers must analyze reports, consider action alternatives, and make the best management decisions based on their personal experience and mental ability to compare the alternatives in mind, rather than taking a systematic and approach to reducing insurance float.

The only alternative is memory-management which is humanly impossible; hence very few practice owners thrive in healthcare and healthcare insurance business.

Accordingly, it is an object of the present invention to provide systems and methods to solve the problems set forth above.

### SUMMARY OF THE INVENTION

It is an object of the embodiments to provide systems and methods to reduce insurance float.

It is an object of the embodiments to provide systems, methods and non-transitory computer readable medium that execute instructions to reduce insurance float. In certain embodiments, the instructions are stored on a memory and are executed by a computer processor.

It is an object of the embodiments to provide systems, methods and non-transitory computer readable medium that execute instructions to provide workflow management and a task list so that staff are able to effectively allocate their resources to process claims and reduce insurance float.

According to a first aspect of the embodiments, a computer-implemented health insurance billing system is provided, comprising: a memory comprising computer executable instructions; and a processor coupled to the memory and configured by the computer executable instructions, the processor configured to:
(1) Access at least one database having patient claim data;
(2) Rank the patient claim data to create a ranked list of patient claim data; and
(3) Generate a list of action items based upon the ranked list of patient claim data, wherein the list of action items are sorted according to Payor float of each of the patient claim data items, wherein steps (2)-(3) are completed on the processor.

In certain embodiments, the computer-implemented health insurance billing system minimizes overall Payor float.

In certain embodiments, the patient claim data is sorted by Payor float of each of the patient claim data items.

In certain embodiments, the Payor float is normalized by a float factor.

In certain embodiments, the float factor includes Payor information, the Payor information sorted by Payor difficulty in making payments and payment delay.

In certain embodiments, the float factor is at least partially sorted by an amount of each of the unpaid patient claim data items.

In certain embodiments, the float factor includes a weighting factor generated by a weighting system, such that the weighting factor can be adjusted based upon Payor data.

In certain embodiments, the system includes heuristic learning, wherein the system is configured to adjust the weighting factor based upon heuristic learning.

In certain embodiments, the float factor is adjusted based upon batch processing such that patient claim data items are sorted into groups and combinations of groups contribute to Payor float.

In certain embodiments, the float factor is configured based upon specific rules, and the rules are followed to create the ranked list of patient claim data.

In certain embodiments, the float factor is adjusted based upon Current Procedural Terminology (CPT) codes and in-network reimbursement fees.

In certain embodiments, the computer-implemented health insurance billing system provides a workflow of action items for each day.

In certain embodiments, the workflow of action items for each day minimizes overall Payor float.

In certain embodiments, the system automatically provides a list of top 10 action items to work on for each day.

In certain embodiments, the system automatically provides a list of action items to work on for each day and delegates them to individual staff at a healthcare institution

In certain embodiments, the system automatically generates a task list on a work bench for multiple users.

In certain embodiments, the at least one database is selected from a group consisting of patients, providers, practice management staff, claims, claim validation rules, tasks, process description, or a combination thereof.

In certain embodiments, the system is in communication with the Payor in order to send claims, appeals, notifications and receive notifications and payments.

Other aspects of the embodiments are achieved by providing a computer-implemented method for minimizes overall Payor float in a health insurance billing system, the method comprising:
(1) Accessing at least one database having patient claim data;
(2) Ranking the patient claim data to create a ranked list of patient claim data; and
(3) Generating a list of action items based upon the ranked list of patient claim data, wherein the list of action items are sorted according to Payor float of each of the patient claim data items, wherein steps (2) and (3) are completed on a processor.

In certain embodiments, step (2) includes a custom software/algorithm based on game theory.

Other aspects of the embodiments are achieved by providing a non-transitory computer readable storage medium storing a computer program product for minimizing Payor float in a healthcare insurance billing system, the non-transitory computer readable storage medium comprising: computer executable instructions and data, the computer executable instructions able to execute a computer program that are able to:
(1) Access at least one database having patient claim data;
(2) Rank the patient claim data to create a ranked list of patient claim data; and
(3) Generate a list of action items based upon the ranked list of patient claim data, wherein the list of action items are sorted according to Payor float of each of the patient claim data items, wherein steps (2)-(3) are performed on a processor.

Other aspects of the invention are directed to a float reduction system comprising: at least one processor communicatively couple to at least one database server; a memory operatively connected to the at least one processor, wherein the memory stores computer executable instructions that, when executed by the at least one processor, causes the at least one processor to execute a method that comprises: accessing the at least one database that includes a plurality of patient claim data items for a healthcare service provider (payee); determining a weighting factor for each of a plurality of patient claim data items for each of a plurality of healthcare insurance providers (payors) to determine a potential float reduction (PFR), wherein the weighting factor takes into account respective payor difficulty in making payments to the payee and heuristic learning of previous attempts to collect payments from each respective payor to the payee, wherein the determined weighting factor is substantially continuously determined; applying the determined weighting factor to each of a plurality of patient claim data items for respective payors to generate a normalized list of patient claim data items; generating a list of action items based upon the normalized list of patient claim data items; sorting the list of action items based on the normalized patient claim data, such that normalized patient claim data is sorted by PFR from highest to lowest; and performing further processing of claims based on the sorted list of action items such that those with the highest PFR are processed soonest, thereby reducing float owed to the payee.

In certain embodiments, the method further involves adjusting the weighting factor based upon batch processing such that patient claim data items are sorted into groups and combinations of groups contribute to payor float.

In certain embodiments, the method further involves configuring the weighting factor based upon specific rules, the rules being followed to create the ranked list of patient claim data.

In certain embodiments, the method further involves adjusting the weighting factor based upon current procedural terminology (CPT) codes and in-network reimbursement fees.

In certain embodiments, the method further involves providing a workflow of action items for each day.

In certain embodiments, the workflow of action items for each day substantially minimizes overall payor float.

In certain embodiments, the method further involves providing a list of action items to work on for each day; and delegating them to individual staff at a healthcare institution.

In certain embodiments, the method further involves generating a task list on a work bench for multiple users.

In certain embodiments, said at least one database is selected from a group consisting of patients, providers, practice management staff, claims, claim validation rules, tasks, process description, or a combination thereof.

In certain embodiments, the method further involves communicating via electronic communication systems with the payor in order to send claims, appeals, notifications and receive notifications and payments.

Other objects of the invention are achieved by providing a method for minimizing overall payor float in a health insurance billing system, the method comprising: accessing at least one database that includes a plurality of patient claim data items for a healthcare service provider (payee), wherein the at least one database is stored in a memory operatively connected to at least one processor, wherein the memory stores computer executable instructions that, when executed by the at least one processor, causes the at least one processor to execute the method, and wherein the at least one database and at least processor comprise the health insurance billing system (system); determining a weighting factor for each of a plurality of patient claim data items for each of a plurality of healthcare insurance providers (payors) to determine a potential float reduction (PFR), wherein the weighting factor takes into account respective payor difficulty in making payments to the payee and heuristic learning of previous attempts to collect payments from each respective payor to the payee, wherein the determined weighting factor is substantially continuously determined; applying the determined weighting factor to each of a plurality of patient claim data items for respective payors to generate a normalized list of patient claim data items; generating a list of action items based upon the normalized list of patient claim data items; sorting the list of action items based on the normalized patient claim data, such that normalized patient claim data is sorted by PFR from highest to lowest; and performing further processing of claims based on the sorted list of action items such that those with the highest PFR are processed soonest, thereby reducing float owed to the payee.

In certain embodiments, the method further involves adjusting the weighting factor based upon batch processing such that patient claim data items are sorted into groups and combinations of groups contribute to payor float.

In certain embodiments, the method further involves configuring the weighting factor based upon specific rules, the rules being followed to create the ranked list of patient claim data.

In certain embodiments, the method further involves adjusting the weighting factor based upon current procedural terminology (CPT) codes and in-network reimbursement fees.

In certain embodiments, the method further involves providing a workflow of action items for each day.

In certain embodiments, the workflow of action items for each day minimizes overall payor float.

In certain embodiments, the method further involves providing a list of action items to work on for each day; and delegating the list of action items to individual staff at a healthcare institution.

In certain embodiments, the method further involves generating a task list on a work bench for multiple users.

In certain embodiments, the at least one database is selected from a group consisting of patients, providers, practice management staff, claims, claim validation rules, tasks, process description, or a combination thereof.

In certain embodiments, the method further involves communicating with the payor via an electronic communications system to send claims, appeals, notifications and receive notifications and payments.

Other objects of the invention are achieved by providing a non-transitory computer readable storage medium storing a computer program product for minimizing payor float in a healthcare insurance billing system, the non-transitory computer readable storage medium comprising computer executable instructions and data, the computer executable instructions able to execute a computer program able to: determine a weighting factor for each of a plurality of patient claim data items for each of a plurality of healthcare insurance providers (payors) to determine a potential float reduction (PFR), wherein the weighting factor takes into account respective payor difficulty in making payments to the payee and heuristic learning of previous attempts to collect payments from each respective payor to the payee, wherein the determined weighting factor is substantially continuously determined; apply the determined weighting factor to each of a plurality of patient claim data items for respective payors to generate a normalized list of patient claim data items; generate a list of action items based upon the normalized list of patient claim data items; sort the list of action items based on the normalized patient claim data items, such that normalized patient claim data is sorted by PFR from highest to lowest; and perform further processing of claims based on the sorted list of action items such that those with the highest PFR are processed soonest, thereby reducing float owed to the payee.

Other aspects of the embodiments and its particular features and advantages will become more apparent from consideration of the following drawings and accompanying detailed description. It should be understood that the detailed description and specific examples, while indicating certain aspects of the embodiments, are intended for purposes of illustration only and are not intended to limit the scope of the embodiments.

### BRIEF DESCRIPTION OF THE DRAWINGS

Aspects of the embodiments are herein described, by way of example only, with reference to the accompanying drawings. With specific reference now to the drawings in detail, it is stressed that the particulars shown are by way of example and for purposes of illustrative discussion of different aspects of the embodiments. In this regard, the description taken with the drawings makes apparent to those skilled in the art how different aspects of the embodiments can be practiced.

FIG. 1 is a block diagram depiction of data flows and processing elements in a computer-implemented method and system for reducing float owed to healthcare service providers according to aspects of the embodiments.

FIG. 2 illustrates a flowchart of a method for assigning tasks to reduce float for the computer system and method as shown in FIG. 1 according to aspects of the embodiments.

FIG. 3 is a flowchart providing various options through which various tasks are performed according to aspects of the embodiments.

FIG. 4 is a flowchart illustrating how tasks are provided to a work bench according to aspects of the embodiments.

FIG. 5 is a flowchart illustrating the determination of probabilities of success for various combinations of actions and staff members according to aspects of the embodiments.

FIG. 6 is a flowchart of a method for a Computer-Assisted Health Insurance Billing To Minimize Payer's Float according to aspects of the embodiments.

FIG. 7 is a flowchart of another method for a Computer-Assisted Health Insurance Billing To Minimize Payer's Float according to aspects of the embodiments.

FIG. 8 is a flowchart of another method for a Computer-Assisted Health Insurance Billing To Minimize Payer's Float according to aspects of the embodiments.

FIG. 9 is a flowchart showing underpayment of an insurance provider such that float is maximized according to aspects of the embodiments.

FIG. 10 illustrates a conceptual block diagram of a computing network environment that can be used to reduce float owed to a healthcare service provider by one or more healthcare insurance companies using the systems and methods described herein according to aspects of the embodiments.

FIG. 11 illustrates a block diagram of the major components of a computer device, personal computer, server, laptop, and/or personal electronic device (herein after, “computer device”) suitable for use to implement the methods shown in regard to FIGS. 1-9, for reducing float using a computer device using the float reduction application(s) and method(s) according to aspects of the embodiments.

FIG. 12 illustrates network system within which the system and method for reducing float owed to a healthcare service provider that can be used by on one or more computer devices can be implemented according to aspects of the embodiments.

FIG. 13 illustrates a flowchart of a float factor based computation including the weighted average of two normalization factors.

### DETAILED DESCRIPTION OF THE INVENTION

In the following description, numerous details are set forth for the purpose of explanation. However, one of ordinary skill in the art will realize that different aspects of the embodiments can be practiced without the use of these specific details.

It is understood that the aspects of the embodiments are not limited to the particular methodology, devices, items, or products etc., described herein, as these may vary as the skilled artisan will recognize. It is also to be understood that the terminology used herein is used for the purpose of describing particular embodiments only and is not intended to limit the aspects of the embodiments. The following exemplary embodiments may be described in the context of exemplary medical devices for ease of description and understanding. However, the aspects of the embodiments are not limited to the specifically described products and methods and may be adapted to various applications without departing from the overall scope of the aspects of the embodiments. All ranges disclosed herein include the endpoints. The use of the term “or” shall be construed to mean “and/or” unless the specific context indicates otherwise.

The embodiments are described more fully hereinafter with reference to the accompanying drawings, in which embodiments of the inventive concept are shown. In the drawings, the size and relative sizes of layers and regions can be exaggerated for clarity. Like numbers refer to like elements throughout. The embodiments can, however, be embodied in many different forms and should not be construed as limited to the embodiments set forth herein. Rather, these embodiments are provided so that this disclosure will be thorough and complete, and will fully convey the scope of the inventive concept to those skilled in the art. The scope of the embodiments is therefore defined by the appended claims.

Reference throughout the specification to “one embodiment” or “an embodiment” means that a particular feature, structure, or characteristic described in connection with an embodiment is included in at least one embodiment of the embodiments. Thus, the appearance of the phrases “in one embodiment” or “in an embodiment” in various places throughout the specification is not necessarily referring to the same embodiment. Further, the particular feature, structures, or characteristics can be combined in any suitable manner in one or more embodiments.

This application incorporates by reference U.S. application Ser. No. 14/665,502 filed on Mar. 23, 2015. The contents of this application are incorporated by referenced herein in their entirety.

The following is a list of the major elements in the drawings in numerical order.
100 Block Diagram Depiction of Data Flows and Processing Blocks/Elements in a Computer-Implemented Method and System to Reduce Float (Method and System)
105 databases
140 Delegation Step
115 Providers
120 Workbench
110 Staff Member
125 New Item
135 Iteration Step/Process
130 Float Calculation Function
145 Evaluation Function
200 Method for Assigning One or More Tasks to Reduce Float for a Payee (Healthcare Service Provider)
210-270 Method Steps of Method 200
302 Process Steps of Establishing a Provider Workbench and Communicating with a Server
304 Databases
306 Case Status List
308 Case(s)
310 Task List
312 Staff Member/Candidates
314 Delegation Process Step
400 Method for Processing a Claim and Generating a Corresponding Float Weight
410-460 Method Steps of Method 400
600 Method for Minimizing Float of a Health Care Provider
610-640 Method Steps of Method 600
700 Method for Minimizing Float of a Health Care Provider
710-750 Method Steps of Method 700
800 Method for Minimizing Float of a Health Care Provider
810-870 Method Steps of Method 800
900 Process of Generating Float by an Insurance Provider
902-912 Process Steps of Process 900
1000 Computing Network Environment
1002 Personal Electronic Device (PED)
1004 PED Float Reduction Task (FRT) Application (FRT App)
1006 Internet/Network (Network)
1008 Float Reduction Master (FRM) Application Server (FRM App Server)
1010 Float Reduction Master (FRM) Application (FRM App)
1012 Database/Storage Server (Storage Server)
1101 Shell/Box
1102 Integrated Display
1104 Internal Data/Command Bus (Bus)
1106 Processor Internal Memory
1108 Processor(s)
1110 Universal Serial Bus (USB) Port
1111 Ethernet Port
1112 Compact Disk (CD)/Digital Versatile Disk (DVD) Read/Write (RW)
1114 Floppy Diskette Drive
1116 Hard Disk Drive (HDD)
1118 Read-Only Memory (ROM)
1120 Random Access Memory (RAM)
1122 Video Graphics Array (VGA) Port or High Definition Multimedia Interface (HDMI)
1124 External Memory Storage Device
1126 External Display
1128 Keyboard
1130 Mouse
1132 Processor Board/PC Internal Memory (Internal Memory)
1134 Flash Drive Memory
1136 CD/DVD Diskettes
1138 Floppy Diskettes
1142 Wi-Fi Transceiver
1144 BlueTooth (BT) Transceiver
1146 Near Field Communications (NFC) Transceiver
1148 Third Generation (3G), Fourth Generation (4G), Long Term Evolution (LTE), Fifth Generation (5G) Cellular Transceiver (Cellular Transceiver)
1150 Communications Satellite/Global Positioning System (Satellite) Transceiver
1152 Antenna
1156 Universal Serial Bus (USB) Cable
1158 Ethernet Cable (CATS Cable)
1160 Printer/Scanner/Facsimile Machine (Printer/Scanner/Fax)
1162 VGA/HDMI Cable
1006 Network System
1202 Mobile Device
1206 Internet Service Provider (ISP)
1208 Modulator/Demodulator (Modem)
1210 Wireless Router
1212 Plain Old Telephone Service (POTS) Provider
1214 Cellular Service Provider
1218 Communication Satellites
1220 Cellular Telecommunications Service Tower (Cellular Tower)
1222 Internet
1224 GPS Station
1226 Satellite Communication Systems Control Station
1228 Global Positioning System (GPS) Satellite

### LIST OF ACRONYMS USED IN THE SPECIFICATION IN ALPHABETICAL ORDER

The following is a list of the acronyms used in the specification in alphabetical order.

### 3G Third Generation

### 4G Fourth Generation

### 5G Fifth Generation

### API Application Programming Interface

### App Executable Software Programming Code/Application

### ASIC Application Specific Integrated Circuit

### BIOS Basic Input/Output System

### BT Bluetooth

### CAP Claim-Action Pair

### CD Compact Disk

### CPT Current Procedural Terminology

### CRT Cathode Ray Tube

### DVD Digital Video Disk

### EEPROM Electrically Erasable Programmable Read Only Memory

### FPGA Field Programmable Gate Array

### FRM Float Reduction Master

### FRT Float Reduction Task

### GAN Global Area Network

### GPS Global Positioning System

### GUI Graphical User Interface

### HDD Hard Disk Drive

### HDMI High Definition Multimedia Interface

### ICAQ Individual Claim Allocation Queue

### ISP Internet Service Provider

### LCD Liquid Crystal Display

### LED Light Emitting Diode Display

### LTE Long Term Evolution

### MODEM Modulator-Demodulator

### NFC Near Field Communications

### PA Partial Allocation

### PC Personal Computer

### PDA Personal Digital Assistant

### PDF Portable Document Form

### PED Personal Electronic Device

### PFR Potential Float Reduction

### POTS Plain Old Telephone Service

### PROM Programmable Read Only Memory

### QPA Queue of Partial Allocations

### QPA Queue of Partial Allocations

### QUB Queue of Unpaid Balances

QUBWA Queue of Unpaid Balances with Actions

### RAM Random Access Memory

### ROM Read Only Memory

### RW Read/Write

### SOP Set-of-Rules

### TAF Total Allocated Float (TAF

### TM Team Member

### USB Universal Serial Bus

### UV Ultraviolet Light

### UVPROM Ultraviolet Light Erasable Programmable Read Only Memory

### VGA Video Graphics Array

### Float

Insurance companies and other Payors (herein after collectively referred to as “Payors”) earn profits in multiple ways. A well-known insurance company business model practice relies on collecting more in premiums than is paid out in claims. A lesser known practice relies on investing money that can be referred to as “float”. Float is the available reserve of funds on hand at any given time; meaning, premiums collected but not yet paid out in claims. Payors capitalize on the fact that they have access to a reserve of funds available between the time they receive payment and when they are to pay out potential claims. The amount of time that the Payor can hold onto said funds is proportional to the return; and therefore, the value of such an asset. The longer the duration, the more return that can be generated using this float.

Not surprisingly, this results in Payors having a disincentive toward paying out claims in a timely manner. Payors enact strict rules and requirements that must be met before any claim funds are paid out. Every delay furthers the Payor's hold onto the float. The slower the pay-out process goes, the more float the Payor accumulates over time. Additional unnecessary delays are caused by the healthcare service provider errors and neglect (herein after, healthcare service providers will be referred to as “Payee(s)”). Payees risk making errors at every stage of their workflow extending the time until a Payee receives payment.

### System and Method Objectives

Reference herein to “a” system and/or “a” method is not to be construed as the aspects of the embodiments being limited to just “one” system and/or just “one” method; that is, there are different aspects of the embodiments of the systems and methods described herein.

It is contemplated that the aspects of the embodiments of the system and method reduces practice management complexity by analyzing patient claims and creating a task list to minimize Payor float.

It is contemplated that the aspects of the embodiments of the system and method identifies and selects staff members to perform tasks in order to reduce Payor float.

It is contemplated that the aspects of the embodiments of the system and method identifies and selects, that is “delegates” tasks to staff members of the Payee, providing them with instructions as to the best next workflow move and necessary information for accomplishing the task more efficiently, and for the best next allocation of tasks resulting in highest presenting the staff member(s) with the next best workflow move for that staff member, and providing only or substantially only the relevant information to make that workflow move a success.

Complexity of healthcare practice management extends beyond human ability to manage it on paper and it grows quickly in step with the addition of new patients. Payors complicate the practice management challenge even more as they introduce an “adversity element” to the overall system because they keep the profit derived from investing the float (the difference between premiums collected and payments made) in financial markets.

The adversity element is, for example, an underpayment or complexity created by a Payor in order to maximize float.

### Complexity Created by Payors

Payors appreciate when Payees make mistakes in processing claims in order to increase Payor float. Payors often do not pay out claims that have spelling mistakes or are not in compliance with the numerous, myriad, and often-times byzantine Payor requirements.

Typical types of complexity created by Payors involve underpaying claims, providing in-network and out of network rates, providing customized group rates and other such payments in order to increase complexity of payments, so as to increase Payor float.

Payors appreciate when the systems and staff of Payees make one or more incorrect choices to increase float, and often increase their own complexity of systems in order to increase float. By increasing float, Payors receive an interest free loan that they are able to invest and keep the profits that are made.

### Advantages of the Systems and Methods According to Aspects of the Embodiments

Aspects of the embodiments involve systems and methods that evaluate float possibility and rank orders by highest float possible. Possibilities are provided in a work bench that staff members use to work on in processing claims; such “work benches” are generally graphical user interfaces (GUIs) that can be used on personal electronic devices (PEDs) as generated by one or more software programs or applications within which the methods described herein can operate, and which are described in greater detail below.

Aspects of the embodiments provides a ranked order of claims to work on and does so automatically, so that staff can target problematic claims and reduce the amount of float.

Aspects of the embodiments system and method are able to handle thousands of claims and unpaid claims and sorts through various choices that leads to high float.

Aspects of the embodiments of the system and method have the following advantages:
1) Understands float;
2) Translates float into a zero sum game;
3) Prioritizes specific action items; and
4) Determines a workflow for a workday (best moves in float minimization game).

Aspects of the embodiments of the system and method involve creating a tree of possible actions and several possibilities of each date. The system computes overall float for the next move (proxy is the float).

In certain aspects of the embodiments, the system and method evaluates the unpaid amount of each claim (e.g., the float), which is the claim value minus the amount paid and calculates the sum of unpaid amounts of each claim (total system float).

Aspects of the embodiments of the system and method sums the options for each claim and provides a potential float reduction (PFR) for each claim—i.e., the total amount of money owed by the Payor to the Payee for a single claim. The items with the highest PFR values are worked on first, so that the overall float of the system is minimized. As those of skill in the art can appreciate, float (e.g., money) owed can range from mere cents to hundreds if not thousands of dollars per claim.

### Float Factor

According to aspects of the embodiments, Payor float can be normalized by a float factor. The float factor can include Payor information, and the Payor information can be sorted by Payor difficulty in making payments and payment delay. According to aspects of the embodiments, the float factor can be at least partially sorted by an amount of each of the unpaid patient claim data items. According to aspects of the embodiments, float factor can include a weighting factor generated by a weighting system, such that the weighting factor can be adjusted based upon Payor data. According to aspects of the embodiments, the float factor can be adjusted based upon batch processing such that patient claim data items are sorted into groups and combinations of groups contribute to Payor float. According to aspects of the embodiments, the float factor can be configured based upon specific rules, and the rules are followed to create the ranked list of patient claim data. According to aspects of the embodiments, the float factor can be adjusted based upon CPT codes and in-network reimbursement fees.

### Weighting Factor

In certain aspects of the embodiments, the system and method calculates and uses a weighting factor for each different Payor in order to calculate a PFR for each item or claim. For example, Medicare is a relatively easy Payor that often pays relatively quickly and completely what is owed, while some insurance companies are difficult Payors that often underpay. In this example, the delay of Medicare is 14 days to transmit payment while others have a 28-day delay.

In certain aspects of the embodiments, the system and method applies a weighting factor to calculate a weighted float for each claim and raises the priority of Payors with longer delay. Thus, the value of each claim is normalized by the weighting factor in order to compute (or generate) a normalized PFR for each claim. Thus, there can be two Payors with identical floats (or money owed) to the Payee, but the Payor whose account is overdue by a longer period of time will be weighted higher than the one that is relatively less overdue; this, of course, incorporates the well-known principle of the time-value aspect of money. Float that is kept by the Payor for longer periods of time means that the Payor has the opportunity to earn more interest and/or investment income on money that is rightfully owed to the Payee, who could then invest or use the money as desired.

### Weighting Factor Calculation

In certain aspect of the embodiments, the system and method includes one or more complex processes in order to compile weighting factors. The system and method takes into account underpayments, types of Payors, In-network vs. Out-of-network rates, and other considerations in order to calculate weighting factors, which is then applied to each claim to obtain a PFR. Each item (or claim) is then ranked by its PFR in order rank the claims, so that a ranked list of claims can be compiled and prioritized and worked on to minimize system float.

### Plan of Action

In certain embodiments, the system involves reviewing a rejected claim to decide what is the next action. The information on hand is the provider's specialty, payer, list of CPT and ICD-10 codes for services delivered, and a charge matching the CPT code.

The system also has a history of all previously paid claims. Each claim has its own CPT and ICD-10 codes, provider specialty and location, and the payer identification, together with its entire history of back-and-forth with the payer, together with the final payment.

From the history, the system compile a database of paths of steps from submission to payment for each combination of specialty, payer, CPT and ICD-10 codes, and location. Each path consists of several pairs of steps: the starting step is the first claim submission, payer responds with a payment or denial, then comes a correction, then again payer response, and so on.

The system wants to get the claim maximally paid in the shortest number of steps. This involves choosing the next step as the step that is the most likely to be paid the most. In case there is a tie (two equal payments), the system selects the one that ends up with the shortest path of steps.

So the system has multiple levels of candidate sequences of steps: possible action, possible responses, again possible action for each response, again possible responses. This tree grows exponentially with each iteration and so explicit computation of every possibility becomes prohibitively expensive.

Float factor estimates the value that is expected to be paid for the given set of CPT codes from this payer in this state given the history from previously paid claims with similar CPT codes from the same payer and the same state.

The history database is never complete and in many cases, the system is unable to find precisely the same already paid claim. So instead, the system will have to find a similar claim, maybe matching only a few of all the matching criteria. The prediction quality improves in step with both the number of claims matching the claim in hand and their own individual matching level. So, for the float factor, the system takes a weighted average of previous payments, where each payment is weighed by its matching weight.

Next, the system computes the possible responses—again each with its own likelihood, which is the fraction of the specific response divided by the total number of responses for the same combination.

The final float weight for each possible action is the sum of the float weights of each subtree from that action.

To expedite the calculation of all possible paths (they grow exponentially), a database of float estimates is created for each combination of codes, specialties, locations and payer. This is the normalization factor for the potential subtree.

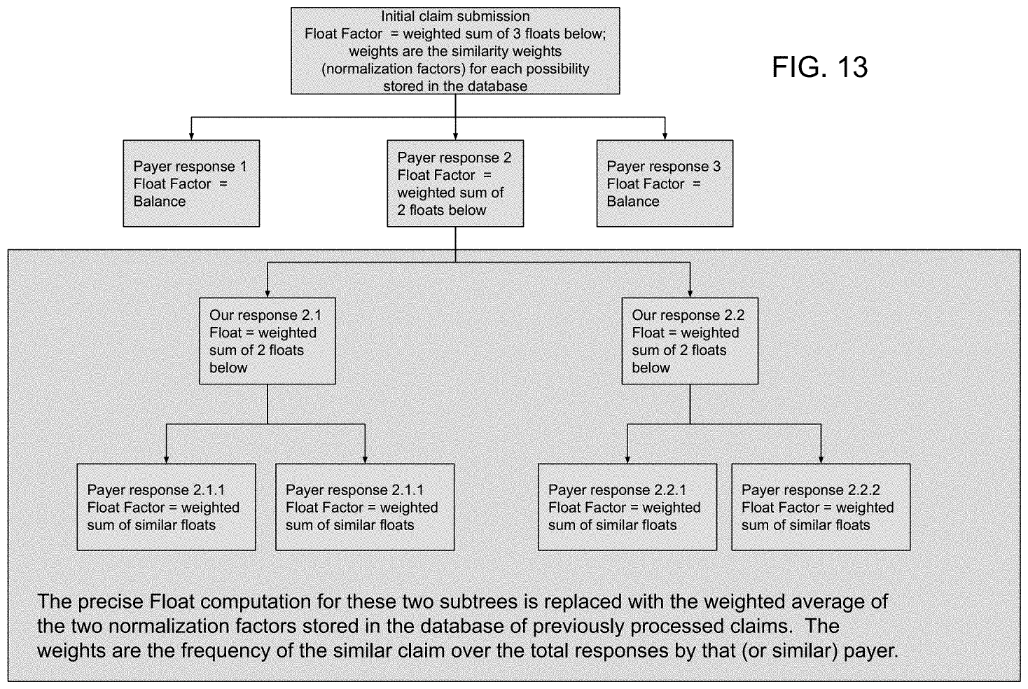

So now instead of computing each Float precisely by computing all of the floats for each of the subtrees, the system takes weighted float estimate which is the weighted average of normalization factors and the weights are the matching weights as before. This is shown, for example in FIG. 13, whereby the precise Float computation for these two subtrees is replaced with the weighted average of the two normalization factors stored in the database of previously processed claims. The weights are the frequency of the similar claim over the total responses by that (or similar) payer.

### Batch Processing

In certain aspects of the embodiments, the system and method groups claims that are unpaid into batches. The system assigns more weight to correct a group of payments, such that if a group of payments are paid it reduces overall system float. As can now be appreciated by those of skill in the art, “system float” refers to a total amount of money kept by one or more Payors that is due to the Payee, or provider of services e.g., medical services provider/facility. In essence, system float can be analogized as a loan made to the Payor, or a debt owed by the Payor to the Payee. It is desirable to reduce system float as much as practically possible.

In an example, if there is a spelling mistake in a patient name, then the claim is not paid. Often spelling mistakes occur in a group of claims so the entire group of claims are unpaid. The system and method provides a weighting factor and assigns a higher weighting factor when correcting a group of payments. The system and method clusters groups of possibilities in a batch process, so that the claims connected to the batch are assigned a higher weighting factor, and thus a higher PFR value.

Heuristic rules can be used to help estimate value of float with specific rules (such as add float together for groups of claims) that associate with cluster as single move. As those of skill in the art can appreciate, in the fields of mathematical optimization and computer science, the term heuristic refers to a technique for solving problems more quickly when classic methods are too slow, or for finding an approximate solution when classic methods fail to find any exact solution. This is achieved by trading optimality, completeness, accuracy, or precision, for speed.

In certain aspects of the embodiments, batch processing of claims helps correct various claims with higher PFR value that have been placed in one or more batches.

### Heuristic Learning

In certain aspects of the embodiments, the system and method described herein, includes heuristic learning, wherein the system is configured to adjust the weighting system based upon heuristic learning. The system and method involves rules, such that the system is able to identify various spelling mistakes and provide a PFR adjustment based upon previously corrected claims.

In other non-limiting examples, the system and method includes machine learning such that the system is able to learn from choices made by human operators, such that successful claims that have been paid can adjust the PFR of each claim, as well as the float factor and weighting factor.

In certain embodiments, the system and method includes various rules to expedite process claims to reduce float. In certain embodiments, there are 2 million rules and there are expedited rules that can be used to help process claims. As such, those of skill in the art can appreciate that systems and methods according to aspects of the embodiments necessarily includes the use of computers and algorithms and associated devices and processing methodologies as the practically innumerable iterations and processes that must be evaluated by definition defies human intervention; that is, the problems solved by the aspects of the embodiments must be solved by computers as no human could possibly know and remember all such rules and processing iterations.

### Underpayment

In certain aspects of the embodiments, the system and method accounts for underpayment of various claims. Underpayment is a delay tactic often employed by Payors in order to partially pay claims, while then keeping the float funds for additional time in order to increase float.

In certain embodiments, the system and method is able to track and focus on underpayments made and increased the PFR based upon underpayments made. This then increases the priority of claims that have been underpaid, thus increasing their PFR and causing these payments to have a higher priority and to be worked on by the staff before other claims with lower PFRs to more effectively and efficiently reduce overall system float.

In certain aspects of the embodiments, the system and method uses machine learning and heuristic learning to identify patterns of underpayment. If a pattern of underpayment is recognized, then the normalized PFR is increased to give priority to those claims with consistent underpayment, and furthermore assigns a higher normalized PFR for those claims with longer periods of under- or no payment.

### Additional Complexity Factors

In certain embodiments, underpayment is a result of use of system codes where Payors only pay up to a certain amount for codes for certain procedures.

Certain Payors include CPT codes and corresponding tables whereby each code is linked to a maximum value paid out by the Payor. As those of skill in the art can appreciate, CPT codes, also known as service codes, are a universal system that identifies medical procedures. Each procedure is given its own unique five digit code that identifies to health insurance companies what type of care was provided. The CPT codes must match the right diagnosis of the patient and the correct procedure and Payors only make payments for claims where all the information is correct.

Adding further complexity to this system is that there are different CPT codes for both in-network and out of network procedures, and Payors often pay different rates for different procedures. Moreover, Payors often strike deals with certain healthcare institutions based upon volume, which leads to additional complexity in codes and payments made.

Additional complexity results from CPT codes having maximum payment amounts associated with them, such that if the healthcare institution charges more than what is allowed according to the respective CPT code, then claims are either underpaid or not paid at all.

### Operation of System and Method

In certain embodiments, the system and method includes a practice position that is defined as a sum of its Payor positions. According to aspects of the embodiments, a position is defined as one or more reactions (or outputs) that can be taken in response to an action or input. That is, in terms of the aspects of the embodiments, in which a medical service provider provides medical care to an insured, the initial action, or position of the Payee (medical service provider), is to submit an initial claim to the appropriate Payor (insurance provider). Because most medical service providers (or Payees) interact with numerous insurance companies (Payors), there are or will be several numerous initial claims submitted; the sum of the Payor positions is the sum of reactions/positions to the initial (and latter resubmissions of) claims submitted to each Payor. As described above, each insurance claim submission includes a float—an amount owed, and the total practice float (or money owed to the Payee) is the sum of the floats for each respective Payor. Every Payor's response (or Payor position) modifies the practice position and its total float. The new position defines a number of potential responses. Each potential response is evaluated using an evaluation function that estimates the resulting float for the potential response and the total float for the Payee. The system:
1) Prunes the list of all possible positions for evaluation (e.g., to determine individual and total float) to a much shorter list of most likely candidate positions to accelerate the position evaluation process, which would be otherwise prohibitively time consuming (that is, because of a very large number of actions that can be taken for any one claim, the system and method according to aspects of the embodiments includes algorithms that can determine those actions (positions) most likely to generate positive responses from Payors);
2) Compares the potential position values on the pruned list and selects the next best position that reduces the float the most; and
3) Places the move that defines that position on the workbench of the staff member that is responsible for that function.

### Figures

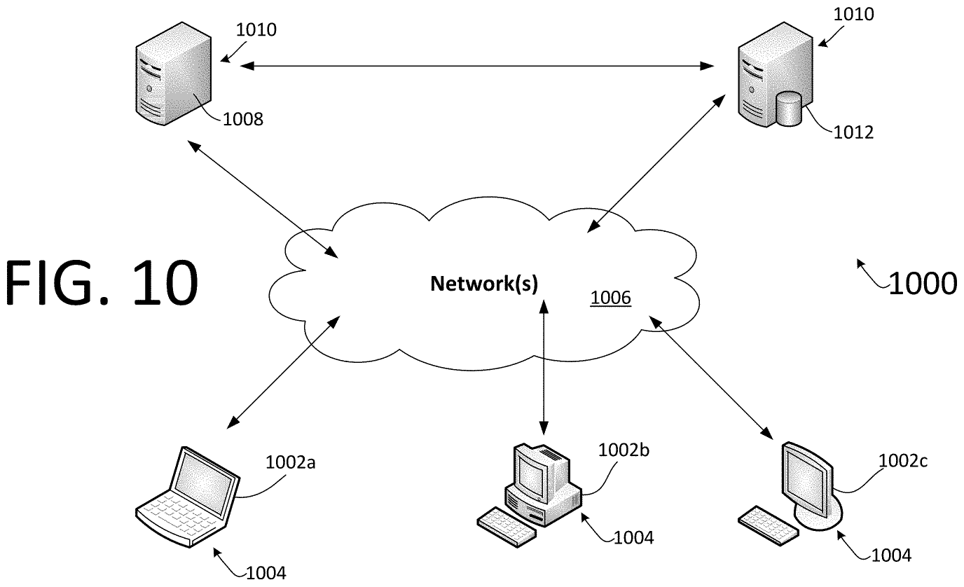

Attention is first directed to FIG. 10. FIG. 10 illustrates a conceptual block diagram of a computing network environment that can be used to reduce float owed to a healthcare service provider by one or more healthcare insurance companies using the systems and methods described herein according to aspects of the embodiments.

The different aspects of the embodiments described herein pertain to the context of systems, methods, and modes for a computer-based health care service provider float reduction application that facilitates the reduction of float owed to the health care service provider by one or more Payors through the processing of information and analysis of responses by a plurality of Payors in response to actions taken by the health care provider to obtain payment from the Payors, but is not limited thereto, except as may be set forth expressly in the appended claims.

As briefly described above, aspects of the embodiments are directed to the reduction of float; float, as described above, is money kept by Payors or insurance companies that is contractually owed to the health care providers, but which is kept by the Payors/insurance companies to generate additional interest income and/or investment income for additional profit by the Payors. Those of skill in the art can appreciate that any insurance industry, including health care insurance, is largely driven and dependent upon actuarial tables and statistics about health care services and costs; insurance companies take in money for premiums, invest some of that money, and know that a certain amount will need to be paid out for claims over any given time period. The investment income generates profits for the insurance company. However, the insurance companies also know that by delaying payment for legitimate claims the money that would otherwise have to be paid out by a first date, can be kept, and invested to generate additional income. The delayed payment duly owed to the health care providers is the float that the aspects of the embodiments seeks to reduce, because float is money owed to the healthcare provided, as further described herein. According to aspects of the embodiments, an application (or a hosted service) can provide float reduction processes and practices.

Uses of the aspects of the embodiments can include one or more of the following, but is not in any limited thereto: health care providers, auto-repair shops, and any other industry that regular submits claims and expects reimbursement for services provided that are covered by, or contractually obligated to, policies under-written by one or more insurance companies.

Aspect of the embodiments substantially solve the problem described above in regard to reducing float that is owed to the Payees.

In FIG. 13, a flowchart is provided whereby the precise Float computation is replaced with the weighted average of the two normalization factors stored in the database of previously processed claims. The weights are the frequency of the similar claim over the total responses by that (or similar) payer.

Aspects of the embodiments substantially reduce practice management complexity by analyzing patient claims and creating a task list to minimize Payor float.

Aspects of the embodiments identify and select staff members of Payees to perform one or more tasks in order to reduce Payor float.

Aspects of the embodiments identify and select, that is “delegate” one or more tasks to staff members, providing them with instructions as to the best next workflow move and necessary information for accomplishing the task more efficiently, and for the best next allocation of tasks resulting in highest probability of payment thereby reducing overall system float owed to the Payee.

Aspects of the embodiments implement one or more servers or computers and one or more software programs or applications, as well as advanced heuristic computer program learning techniques, to reduce float according to the processes described herein. It is known to those of skill in the art that any one healthcare service provider may have to have knowledge of as many as about 100,000 procedure codes and about 100,000 diagnostic codes, and such codes must match the services provided in order for the Payors to pay the insurance claim. In addition, there are numerous rules that must be adhered to in submitting insurance claims for any one healthcare insurance provider. Further, those of skill in the art can appreciate that there are over 2,000 health care insurance providers in the United States, and that each one will have their own particular set of rules in regard to insurance claims. Still further, it is known to those of skill in the art that even in regard to a single health care insurance provider, different states will have different laws, rules, and regulations that must be adhered to in order for the insurance company to properly process and pay the insurance claim. As a result, those of skill in the art can appreciate that there are, literally, millions of iterations of insurance claims that a healthcare service provider can submit to insurance companies; consequently, such processing cannot be accomplished by humans without the assistance of programs and heuristic learning techniques to not only properly fill out the insurance claim forms but to also predict possible reactions by the insurance companies in denying payment of the insurance claims, thereby increasing float.

In the following detailed description, references are made to the accompanying drawings that form a part hereof, and in which are shown by way of illustrations, specific embodiments, or examples. These aspects may be combined, other aspects may be utilized, and structural changes may be made without departing from the spirit or scope of the present disclosure. The following detailed description is therefore not to be taken in a limiting sense, and the scope of the present invention is defined by the appended claims and their equivalents.

While some embodiments will be described in the general context of program modules that execute in conjunction with an application program that runs on an operating system on a personal computer, those skilled in the art will recognize that aspects may also be implemented in combination with other program modules.

Generally, program modules include routines, programs, components, data structures, and other types of structures that perform particular tasks or implement particular abstract data types. Moreover, those of skill in the art can appreciate that different aspects of the embodiments can be practiced with other computer system configurations, including hand-held devices, multiprocessor systems, microprocessor-based or programmable consumer electronics, minicomputers, mainframe computers, and comparable computing devices. Aspects of the embodiments can also be practiced in distributed computing environments where tasks are performed by remote processing devices that are linked through a communications network. In a distributed computing environment, program modules can be located in both local and remote memory storage devices.

Aspects of the embodiments can be implemented as a computer-implemented process (method), a computing system, or as an article of manufacture, such as a computer program product or computer readable media. The computer program product can be a computer storage medium readable by a computer system and encoding a computer program that comprises instructions for causing a computer or computing system to perform example process(es). The computer-readable storage medium is a computer-readable memory device. The computer-readable storage medium can for example be implemented via one or more of a volatile computer memory, a non-volatile memory, a hard drive, a flash drive, a floppy disk, or a compact disk, and comparable hardware media.

Throughout this specification, the term “platform” can be a combination of software and hardware components for providing share permissions and organization of content in an application with multiple levels of organizational hierarchy. Examples of platforms include, but are not limited to, a hosted service executed over a plurality of servers, an application executed on a single computing device, and comparable systems. The term “server” generally refers to a computing device executing one or more software programs typically in a networked environment. More detail on these technologies and example operations is provided below.

A computing device, as used herein, refers to a device comprising at least a memory and one or more processors that includes a server, a desktop computer, a laptop computer, a tablet computer, a smart phone, a vehicle mounted computer, or a wearable computer. A memory can be a removable or non-removable component of a computing device configured to store one or more instructions to be executed by one or more processors. A processor can be a component of a computing device coupled to a memory and configured to execute programs in conjunction with instructions stored by the memory. Actions or operations described herein may be executed on a single processor, on multiple processors (in a single machine or distributed over multiple machines), or on one or more cores of a multi-core processor. An operating system is a system configured to manage hardware and software components of a computing device that provides common services and applications. An integrated module is a component of an application or service that is integrated within the application or service such that the application or service is configured to execute the component. A computer-readable memory device is a physical computer-readable storage medium implemented via one or more of a volatile computer memory, a non-volatile memory, a hard drive, a flash drive, a floppy disk, or a compact disk, and comparable hardware media that includes instructions thereon to automatically save content to a location. A user experience can be embodied as a visual display associated with an application or service through which a user interacts with the application or service. A user action refers to an interaction between a user and a user experience of an application or a user experience provided by a service that includes one of touch input, gesture input, voice command, eye tracking, gyroscopic input, pen input, mouse input, and keyboards input. An application programming interface (API) can be a set of routines, protocols, and tools for an application or service that allow the application or service to interact or communicate with one or more other applications and services managed by separate entities.

While example implementations are described using personal computer or computer server applications herein, embodiments are not limited to such implementations. As discussed previously, the aspects of the embodiments are directed to the reduction of float due to a healthcare service provider. An organizational structure of an application according to embodiments are not limited to the examples discussed herein.

Technical advantages exist for determining how to reduce float utilizing the aspects of the embodiments. Such technical advantages include using computer implemented heuristic methods and algorithms to learn and keep track of past actions and outcomes, and determining probabilistic outcomes of thousands if not tens or even hundreds of thousands of possible combinations of actions, re-actions, and staff members to execute such actions. Such technical advantages can further include accessing one or more databases that retain a substantially large amount of data including the rules, laws, regulations for each of over two-thousand different healthcare insurance providers, and wherein such rules, laws, and regulations can change for the same health care insurance provided on a state-by-state basis. The processing of such vast amounts of data cannot, literally, be accomplished by staff members within any reasonable amounts of time in a business environment. Further, such technical advantages include the processing of claims according to aspects of the embodiments substantially completely using computers and communicating such processed claims to each of the healthcare insurance providers in an electronic format that is required by such insurance providers, ostensibly to improve processing times and reduce costs.

Aspects of the embodiments address a need that arises from very large scale of operations created by networked computing and cloud-based services that cannot be managed by humans. The actions/operations described herein are not a mere use of a computer, but address results of a system that is a direct consequence of software used as a service such as communication services offered in conjunction with communications.

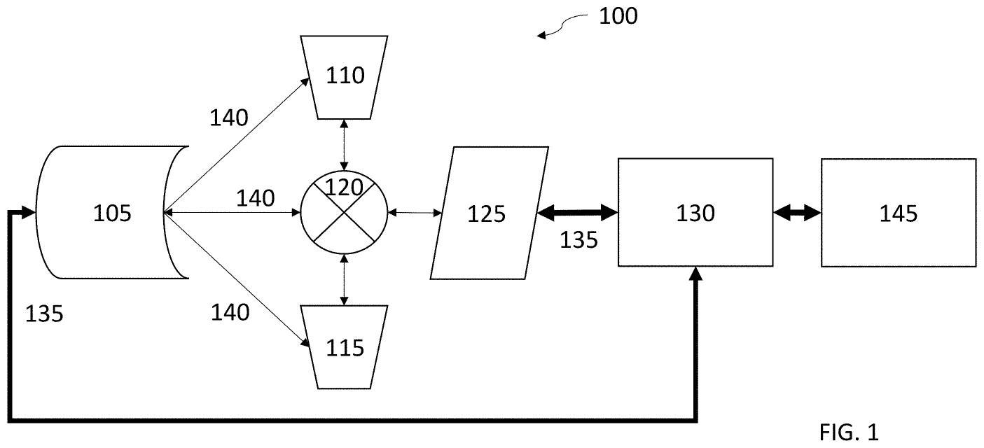

FIGS. 1-9 illustrate various aspects of float reduction program or application for use on one or more computing devices, including, according to certain aspects of the embodiments, use of the internet or other similar networks. The float reduction program provides a practical, technical solution to the problem of reducing float owed to healthcare providers; as those of skill in the art can appreciate, the aspects of the embodiments have no “analog equivalent” as its embodiments reside solely or substantially in the physical device or computer domain. That is, the ability to try and reduce float, with over 2,000 healthcare insurance providers, operating with or under different rules, laws, and regulations depending on jurisdiction and insurance provider, has always meant, and continues to mean, using practical, non-abstract physical devices such as computers. Further such insurance providers require computerized/electronic communications. The technological improvement of the aspects of the embodiments resides in at least in the ability to quickly and easily integrate information from a plurality of sources, but also in determining, using one or more algorithms and/or computerized heuristic learning processes to rank possible actions to new tasks based on previous responses to the same or similar tasks, and/or to take new rules, laws, and regulations into account. Thus, such processing can only be accomplished in a manner that can only be done on a computer, and it allows a user to manipulate, process, and use the relevant information none of which could be accomplished without a computer or some other technological equivalent.

Referring now to FIG. 10, float reduction master (FRM) application (App) server host (FRM App server) 1008 can execute FRM application (FRM App) 1010 that alone, or in combination with personal electronic device (PED) float reduction task (FRT) application (PED FRT App) 1004 (used and stored in PEDs 1002a-c) provides the capability to reduce float. Computing network environment 1000 can also include database/storage server (database server) 1012, which can enable storing and processing of claims among users (not shown). Database server 1012 can interface with FRM App server 1008 directly or through Internet/network (network) 1006. Network 1006 can also interface with PEDs 1002a-c with FRM App server 1008 according to aspects of the embodiments. Original or processed claims, as well as tasks (described in greater detail below) and the results of heuristic learning processes/algorithms can be stored in one or more data stores (for example, local data stores in users' PEDs, cloud storage, and so on), some of which may be managed by a database server 1012. The content and associated data may be managed by multiple servers. Similarly, FRM App 1010 (or hosted service) can be executed on other multiple servers as well.

Users can access either or both of FRT App 1004 and FRM App 1010 through PEDs 1002a-c. As those of skill in the art can appreciate, the float reduction application can be embodied as either a sold or licensed stand-alone software product, or it can be sold or licensed and embodied in the form as shown in FIG. 10, that is, a portion on PEDs 1002a-c and a portion on FRM Server 1008; for the purposes of this discussion, from hereon in, the float reduction application will be presumed to be adapted to be in the latter configuration, but both applications will be referred to as float reduction App 1004/1010 in fulfillment of the dual purposes of clarity and brevity.

Network 1006 can be one or more different or separate networks, and can provide wired or wireless communications between nodes, such as PEDs 1002a-c, or servers 1008, 1012. According to aspects of the embodiments, float reduction App 1004/1010 can also be locally executed on a user's computing device e.g., PED 1002a-c. To process the claims and enable communication the same to insurance providers, float reduction App 1004/1010 can provide a user experience to the users. The user experience can be a visual display through which the users can interact with float reduction App 1004/1010. The interactions can include a touch input, a gesture input, a voice command, eye tracking, a gyroscopic input, a pen input, mouse input, and/or a keyboards input, among others. As discussed in further detail below, the user experience can provide visual indications of sharing status of content such as documents, portions of documents, collations of documents, etc.

PEDs 1002a-c can each include a display device, such as a touch enabled display component, and a monitor, among others, to provide access to float reduction App 1004/1010 for the users through a web browser (thin client) or a local client application (thick client). PEDs 1002a-c can include a desktop computer, a laptop computer, a tablet, a handheld device, a vehicle mount computer, an embedded computer system, a smart phone, and a wearable computer, among other computing devices, for example.

While computing network environment 1000 as illustrated in FIG. 10 has been described with specific components including servers 1008, 1012, PEDs 1002a-c, network 1006, float reduction App 1004/1010, aspects of the embodiments are not limited to these components or system configurations and can be implemented with other system configuration employing fewer or additional components. Attention is now directed to FIG. 1, which is a block diagram depiction of data flows and processing elements in a computer-implemented method and system to reduce float (method and system) 100 of according to aspects of the embodiments. Method and system 100 comprises a plurality of databases, depicted as databases 105, such as a patient databases, a provider database, a practice management staff database, claims database, claim validation rule database, tasks database, and process descriptions (policies, flowcharts, and checklists), among other processing steps and system elements.

As shown, each staff member 110 and each provider 115 has an individual task and claim workbench 120, which must be periodically checked for presence of new items 125. According to aspects of the embodiments, new item 125 can include a new claim, or new task related to a new or previous claim, among other things to do or accomplish. According to aspects of the embodiments, new item 125 can also include bi-directional connectivity and communication with and to Payors to send claims, appeals, receive notifications, authorize/send payments, and the like. Claim workbench 120 can be embodied in the form of a graphical user interface (GUI) that can be accessed via PED 1002 and operation of float reduction App 1004/1010.

In Example of a GUI is U.S. D860,239 entitled Display screen with graphical user interface for medical billing workflow management, which issued Sep. 17, 2019. The contents of this application are incorporated by reference into this application in their entirety.

A selection or delegation algorithm 130 continuously iterates 135 through all the databases 105 and delegates 140 the best workflow action for each workbench 120.

According to aspects of the embodiments, a practice position is defined as a sum of its Payor positions. According to aspects of the embodiments, the total practice float is the sum of the floats for each Payor. According to aspects of the embodiments, every Payor's variable response modifies a practice position and its respective float. According to aspects of the embodiments, the new practice position defines a number of potential responses. According to aspects of the embodiments, each potential response is evaluated using an evaluation function 145 that estimates the resulting float of the entire practice position across each workbench 120.

According to aspects of the embodiments, the float calculation function 130 via the evaluation function 15 compares the potential position values on the pruned list of possible positions for evaluation, and identifies and then selects the next best position that most reduces the float. As discussed above, decreasing float is essentially the same as decreasing the amount of money that is owed to the practice or Payee (medical service provider) because it is money that has been earned but not yet paid to the Payee.

According to aspects of the embodiments, the float calculation function 130 prunes the list of all possible positions for evaluation (float) to a much shorter list of most likely staff member(s) 110 and/or provider(s) 115 to accelerate the position evaluation process 120, which would be otherwise prohibitively time consuming, and places the workflow move that defines that position in the workbench 120 of the staff member 110 responsible for that function.

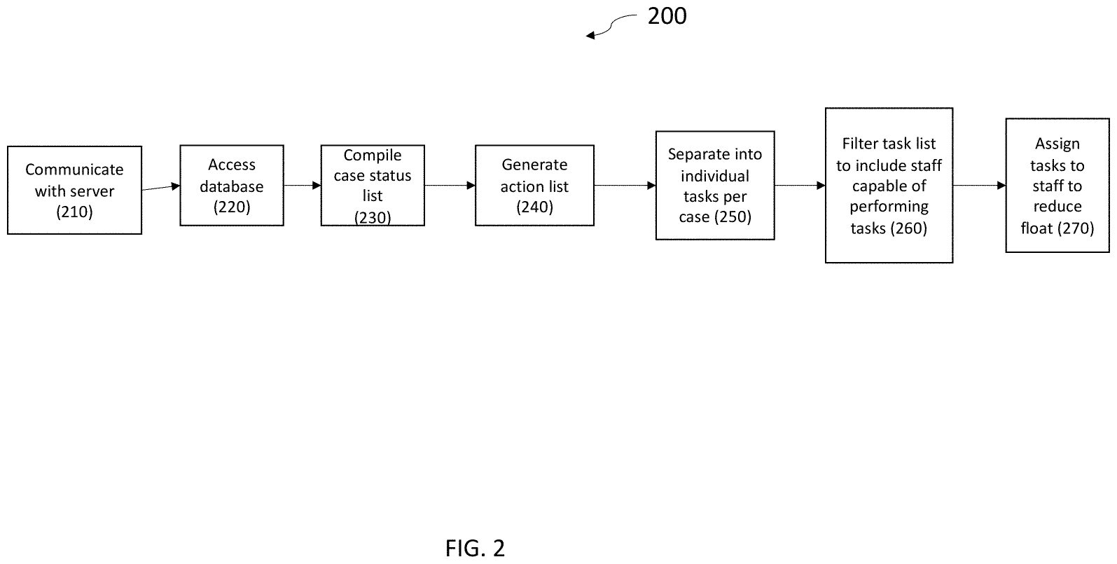

FIG. 2 illustrates a flowchart of a method for assigning one or more tasks to reduce float for a Payee using the computer system and method as shown in FIG. 1 (method 200) according to aspects of the embodiments. Method 200 begins with method step 210 in which float reduction App 1004/1010 communicates with one or more servers 1002, 1010, or 1012, and in method step 220, method 200 accesses one or more databases 105. In method step 230, method 200 compiles a case status list 230.

According to aspects of the embodiments the following is a non-limiting list of examples of what a case can be: a case can be a single claim, a plurality of claims for a single patient, a plurality of claims for a group of people related by blood/marriage to each other, a plurality of claims according to medical procedure, one or more claims based on Payor (e.g., all of the claims supposed to be paid by Insurance Co. “XYZ”), a plurality of claims based on age, a plurality of claims based on an amount of float, a group of claims based on probability of resolution within a certain time period (e.g., a first set of claims thought to have a 90% chance of being resolved in two months or less, a second set of claims thought have a 70% chance of being resolved in four months, and so on), among other manners of grouping claims. Thus, the compiling of the case status list can identify those cases that have the greatest float, or the oldest outstanding unresolved claims, or a combination thereof, among other manners of compiling and prioritizing.

In method step 240, method 200 generates an action list. According to aspects of the embodiments, an action list can be an “action item list” or, more simply, a “to-do” list. That is, in method step 240 method 200 and float reduction App 1004/1010 executes further instructions in a memory in order to generate a ranked list of cases in order to reduce the overall system float of the system.

Method 200 then proceeds to method step 250 in which each case is assigned a list of tasks to perform. That is, to process each claim with outstanding float, there may be one or more tasks that need to be performed (e.g., correct spelling, correct the name of the patient, obtain a better medical procedure code, among other tasks) into individual cases. In method step 260 method 200 filters the task list to include all staff members that can perform the task, and in method step 270 method 200 assigns tasks to staff members to reduce float.

In certain embodiments, the tasks are created and assigned that are spread across different claims. For example, a first person has the task(s) of correcting spelling in names for many different claims and a second person has the task(s) of verifying/correcting medical procedure codes for many different claims. This is shown in D847,170 entitled Display screen with graphical user interface for practice workflow management, which is incorporated by reference in its entirety into this application.

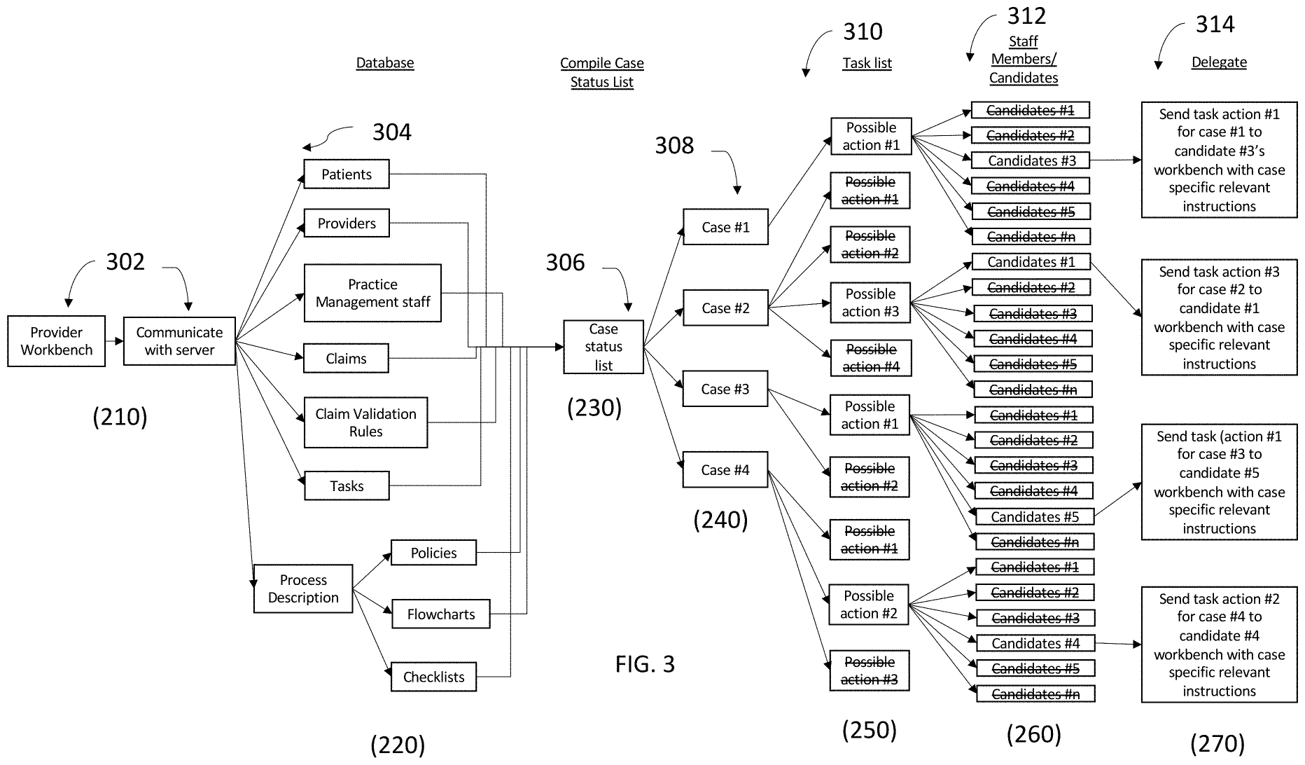

FIG. 3 shows a workbench and a task list showing various databases and action items and compiling a task list of cases and items across a workbench to various staff members/candidates to complete the tasks. The system complies the workflow and uses rules for claim validation and weighting factors in order to assign the tasks to staff/candidates for task completion in an organized and simplified manner. That is, according to aspects of the embodiments, FIG. 3 illustrates the results of the steps of method 200 shown in FIG. 2, as annotated in FIG. 3. Thus, method step 210 pertains to the blocks 302; method step 220 pertain to blocks 304; method step 230 pertain to block 306; method step 240 pertains to blocks 308; method step 250 pertains to blocks 310; method step 260 pertains to blocks 312; and method step 270 pertains to blocks 314.

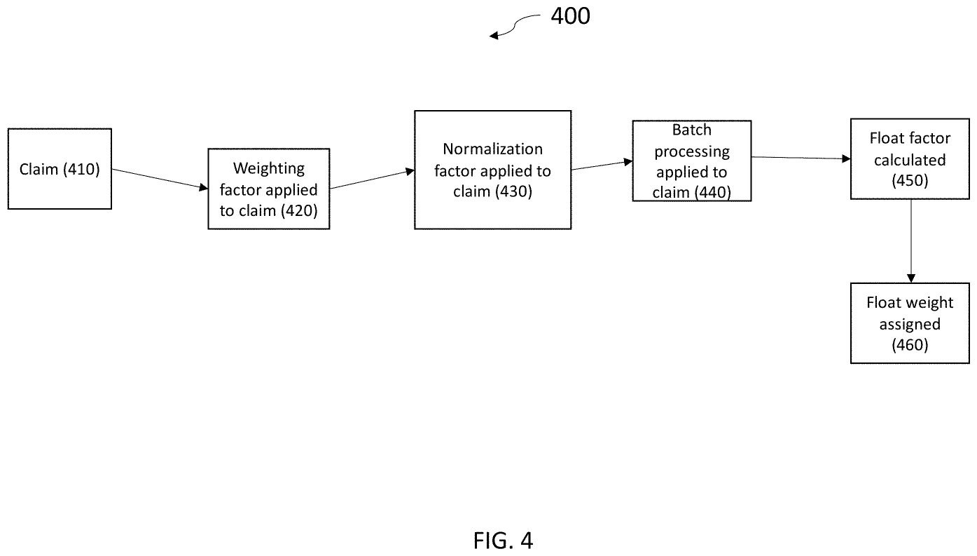

FIG. 4 illustrates a flowchart of a method for processing a claim and generating a corresponding float weight by float reduction App 1004/1010 according to aspects of the embodiments. Method 400 begins with method step 410 in which a claim is retrieved from memory, and in method step 420 method 400 generates a weighting factor 420. In method step 430 a normalization factor is generated for the claim, and in method step 440 a batch processing factor is generated, all of which are used to calculate a float factor in method step 450. The claim is then provided or assigned a float weight in method step 460, and then the claim is placed in a ranked list according to float using the determined values of weighting factor, normalization factor, batch processing factor, float factor and weighting factor according to aspects of the embodiments.

In certain embodiments, the Float (tree)=Float of root action+Sum of (Float (subtree i)×Weight of subtree i)

Float of root action=sum of (Float for similar claim(i)×normalization factor(i))
Weight of subtree=Sum of (frequency of the similar claim over total responses by the payer)

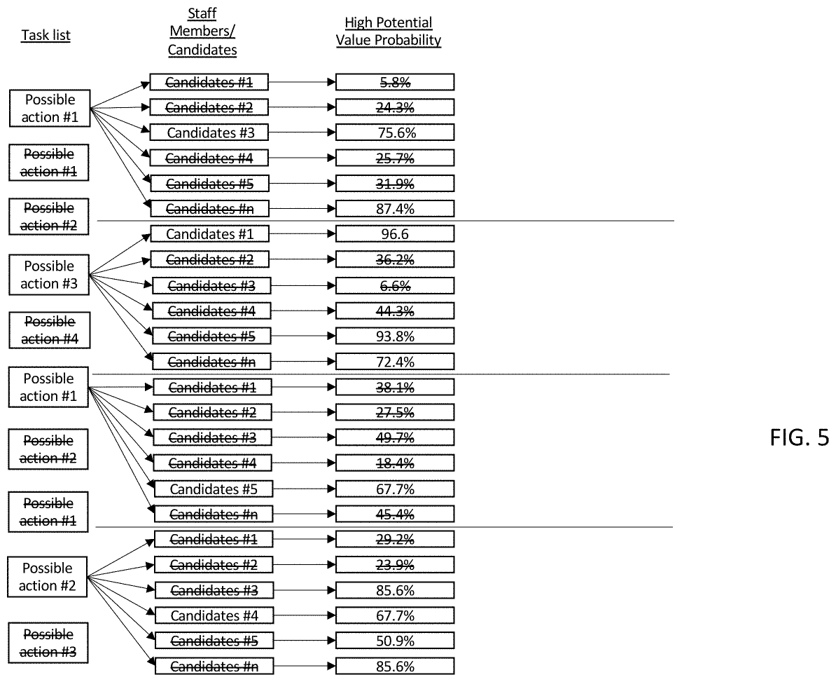

FIG. 5 shows a chart providing various tasks and possible actions for a limited set of tasks for a single claim, and a sorting of the tasks based upon the potential value/probability to reduce float. Referring to FIG. 5, there are, in this non-limiting example, four possible tasks or actions that can be taken (“Possible Action #1-#4). Possible Action #1 502a can be assigned to six different candidates, and each has a separate potential value/probability, ranging from 5.8% to 87.4%. Another task, Possible Action #3 502b, can be assigned to six different candidates, and each of these also has a separate potential value/probability, ranging from 6.6% to 96.6%, and so on.

As shown, the highest probability takes are not crossed out and are the tasks that the system handles first in order to reduce float.

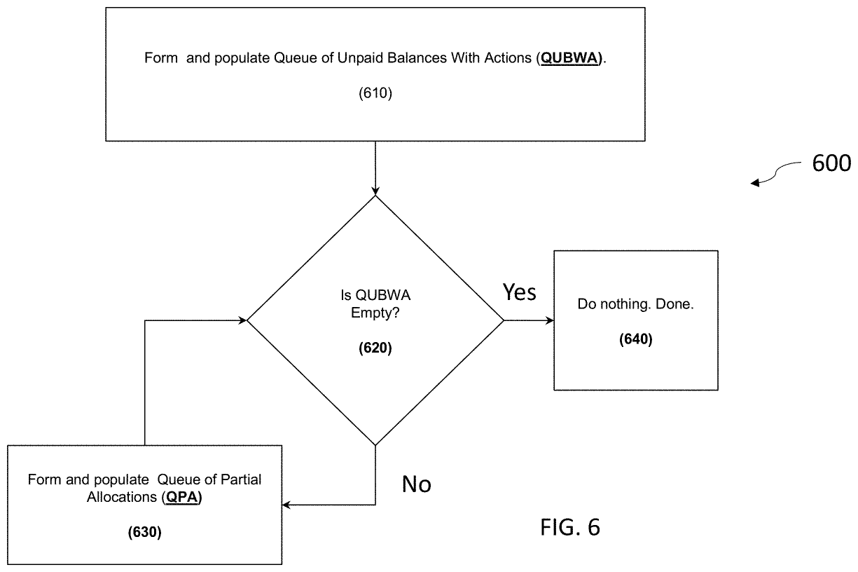

FIG. 6 is a flowchart of method 600 for minimizing float of a health care provider using a computer implemented system according to aspects of the embodiments. Method 600 can be implemented through use of float reduction App 1004/1010 in PED 1002, FRM App Server 1008, and database server 1012 according to aspects of the embodiments.

In method step 610 float reduction App 1004/1010 forms and populates a queue of unpaid balances with actions (QUBWA). In method step 620, float reduction App 1004/1010 checks to see if the QUBWA is empty. If the QUBWA is empty (“Yes” path from decision step 620), then float reduction App 1004/1010 does nothing and returns to method step 610 the next time method 600 is implemented. If the QUBWA is not empty (“No” path from decision step 620), then float reduction App 1004/1010 causes method 600 to method step 630, wherein float reduction App 1004/1010 forms and populates a queue of partial allocations (QPA), and returns to decision step 620. A QPA is a list of claims or a list of groups of claims that have float associated with them. According to aspects of the embodiments, one manner of ranking is simply by monetary value alone, or by length of time the claim is outstanding, or by a combination thereof, with one or the other of length of time/monetary values weighted, among other manners of ranking (such as ease of dealing with a Payor). This process is reiterated until a ranked task list is created.

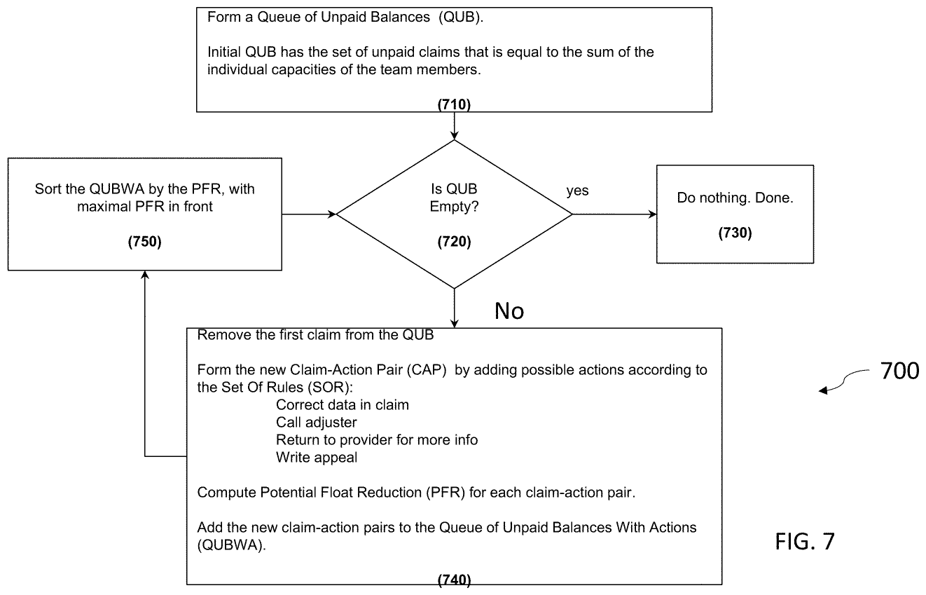

FIG. 7 is a flowchart of method 700 for minimizing float of a health care provider using a computer implemented system according to aspects of the embodiments. Method 700 can be implemented through use of float reduction App 1004/1010 in PED 1002, FRM App Server 1008, and database server 1012 according to aspects of the embodiments.

Method 700 begins with method step 710 in which float reduction App 1004/1010 forms a queue of unpaid balances (QUB). The initial QUB comprises a set of unpaid claims that is equal to the sum of the individual capacities of the team members.

In certain embodiments, At the start of the workshift, each team member is given 40 claims along with the best actions suggested by the system. As time goes on, different team members will complete their initial assignments at different times and become idle. So the next claim along with the computer-selected action will go to the team member who has been idle the longest.

In decision step 720, float reduction App 1004/1010 checks to see if the QUB is empty and if yes (“Yes” path from decision step 720), method 700 proceeds to step 730 wherein no further activity occurs (or returns to step 710 the next time method 700 is implemented). Otherwise, in “No” path from decision step 720, method 700 as implemented by float reduction App 1004/1010 removes the first claim from the QUB. A new claim-action pair (CAP) is formed by adding possible actions according to the set-of-rules (SOR). The SOR can include one or more of the following rules: correct data in claim; call an adjuster; return the claim to the provider for more info; write an appeal, among other rules. Method step 740 the computes a PFR for each claim-action pair according to aspects of the embodiments. Float reduction App 1004/1010 in method 700 then adds new claim-action pairs to the queue of unpaid balances with actions (QUBWA). Following method step 740, method 700 proceeds to method step 750, in which system and float reduction App 1004/1010 sorts the QUBWA by the PFR with the QUBWA with the maximum PFR first in the list. Following method step 750, method 700 and float reduction App 1004/1010 returns to decision step 720 to reiterate the process until a substantially complete list of ranked items is created.

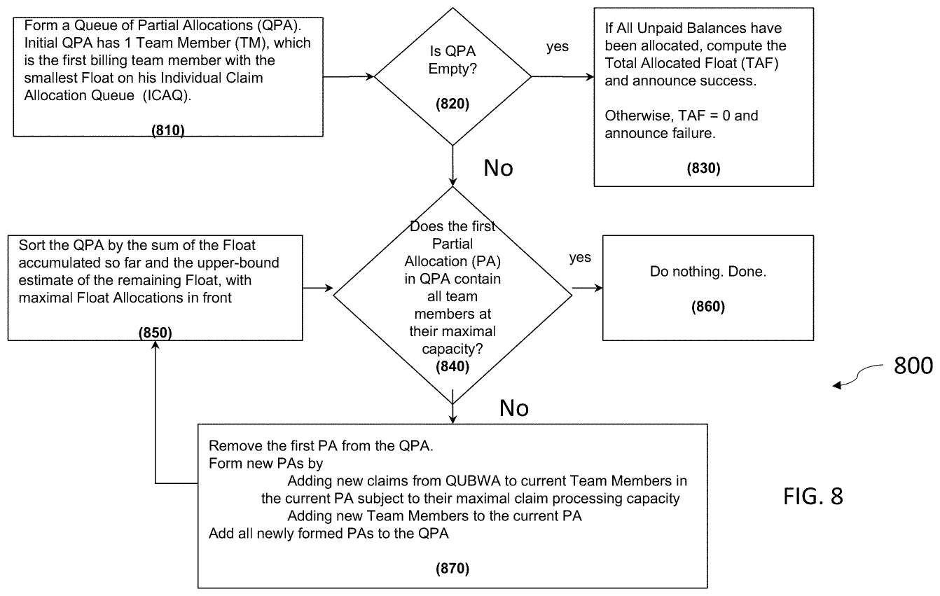

FIG. 8 is a flowchart of method 800 for minimizing float of a health care provider using a computer implemented system according to aspects of the embodiments. Method 800 can be implemented through use of float reduction App 1004/1010 in PED 1002, FRM App Server 1008, and database server 1012 according to aspects of the embodiments.

Method 800 begins with method step 810 in which float reduction App 1004/1010 forms a queue of partial allocations (QPA). The initial QPA has 1 team member (TM), which is the first billing team member with the smallest float on their individual claim allocation queue (ICAQ).

In decision step 820, method 800 and float reduction App 1004/1010 determines whether the QPA is empty. If the QPA is empty (“Yes” path from decision step 820), then the system and float reduction App 1004/1010 checks to see if all unpaid balances have been allocated (method step 830), computes the total allocated float (TAF), and announce success. If there are un-allocated and unpaid balances, then TAF is set equal to 0 and a failure is announced.

If, however, the QPA is not empty (“No” path from decision step 820), method 800 and float reduction App 1004/1010 checks to see if the first partial allocation (PA) in the QPA contains all team members at their maximal capacity (in decision step 840) If yes (“Yes” path from decision step 840), then the system and method as implemented via float reduction

App 1004/1010 does nothing (method step 860). If, however, the QPA does not contain all team members at their maximal capacity (“No” path from decision step 840), then in method step 870 the system and float reduction App 1004/1010 removes the first PA from the QPA and forms new PAs by: adding new claims from QUBWA to current TMs in the current PA subject to their maximal claim processing capacity; adding new TMs to the current PA; and adding all newly formed PAs to the QPA. Method 800 then proceeds to method step 850 in which float reduction App 1004/1010 and the system sorts the QPA by the sum of the float accumulated so far and the upper-bound estimate of the remaining float, with maximal float allocations in front. Following method step 850, method 800 returns to decision step 840, to again determine if the first PA in the QPA contains all team members at their maximal capacity, and method 800 continues to repeat steps 840, 870, and 850 until all of the team members are operating at their maximal capacity.

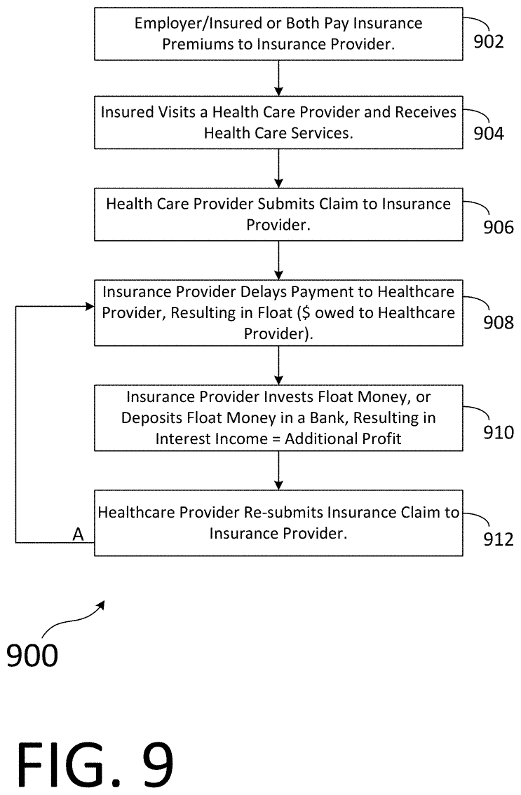

FIG. 9 is a flowchart of process 900 that shows how underpayment by an insurance provider occurs, resulting in float. Process 900 begins with block 902 in which an insured, employer of the insured, or both, pay insurance premiums to a health care insurance provider. In block 904 the insured visits a healthcare provider and receives care. In block 906 the healthcare provider then submits an initial insurance claim to the insurance company. In block 908, the insurance company delays payment to the healthcare provider, resulting in float, or money owed to the healthcare provider. In block 910 the insurance provider can invest that money, or deposit it into a savings account to earn interest. Any money that earns interest, or appreciates in value through investments is additional profit for the insurance provider. In block 912 the healthcare provider re-submits the claim to the insurance company following path A; sometimes the insurance companies pay the claims promptly, after a second submission, or more often they do not, and the paths of blocks 908-912 are repeated.

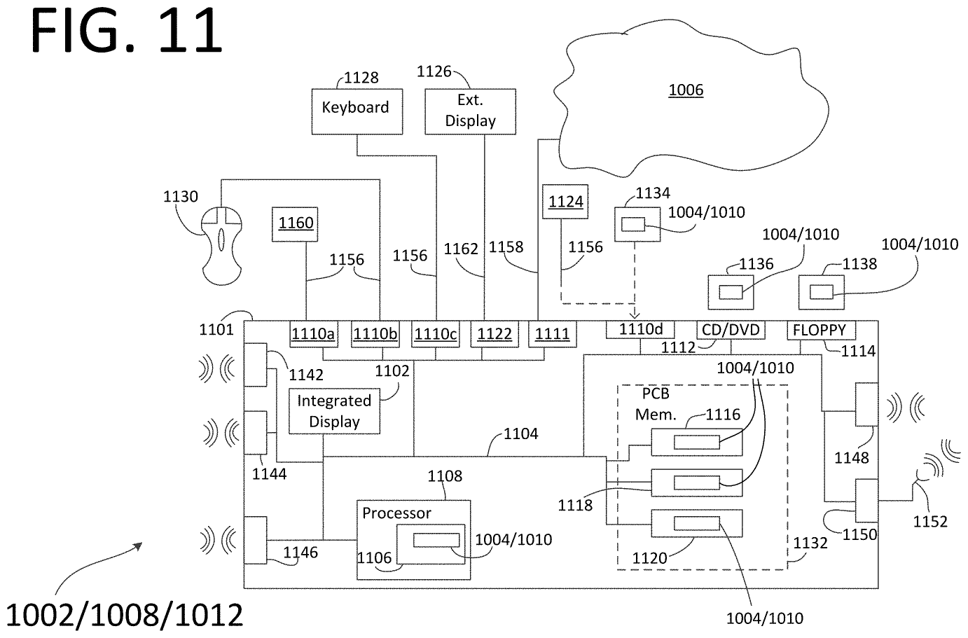

FIG. 11 illustrates a block diagram of the major components of a computer device, PC, server, laptop, and/or PED 102, 108, 112 (herein after, “computer device”) suitable for use to implement the methods and processes shown in FIGS. 1-9 for reducing float using one or more computer devices according to aspects of the embodiments. The computer device comprises, among other items, shell/box 1101, integrated display/touch-screen 1102 (though not used in every application of the computer device), internal data/command bus (bus) 1104, processor board/PC internal memory (internal memory) 1132, and one or more processors 1108 with processor internal memory 1106 (which can be typically read only memory (ROM) and/or random access memory (RAM)). Those of ordinary skill in the art can appreciate that in modern computer device systems, parallel processing is becoming increasingly prevalent, and whereas a single processor would have been used in the past to implement many or at least several functions, it is more common currently to have a single dedicated processor for certain functions (e.g., digital signal processors) and therefore could be several processors, acting in serial and/or parallel, as required by the specific application. The computer device further comprises multiple input/output ports, such as universal serial bus (USB) ports 1110, Ethernet ports 1111, and video graphics array (VGA) ports/high definition multimedia interface (HDMI) ports 1122, among other types. Further, the computer device includes externally accessible drives such as compact disk (CD)/digital versatile disk (DVD) read/write (RW) (CD/DVD/RW) drive 1112, and floppy diskette drive 1114 (though less used currently, some computer devices still include this type of interface). The computer device still further includes wireless communication apparatus, such as one or more of the following: Wi-Fi transceiver 1142, BlueTooth (BT) transceiver 1144, near field communications (NFC) transceiver 1146, third generation (3G)/fourth Generation (4G)/long term evolution (LTE)/fifth generation (5G) transceiver (cellular transceiver) 1148, communications satellite/global positioning system (satellite) transceiver 1150, and antenna 1152.

Internal memory 1132 itself can comprise hard disk drive (HDD) 1116 (these can include conventional magnetic storage media, but, as is becoming increasingly more prevalent, can include flash drive memory 1134, among other types), ROM 1118 (these can include electrically erasable programmable ROM (EEPROMs), ultra-violet erasable PROMs (UVPROMs), among other types), and RAM 1120. Usable USB port 1110 is flash drive memory 1134, and usable with CD/DVD/RW drive 1112 are CD/DVD diskettes (CD/DVD) 1136 (which can be both read and write-able). Usable with floppy diskette drive 1114 are floppy diskettes 1138. External memory storage device 1124 can be used to store data and programs external to box 1101 of the computer device, and can itself comprise another hard disk drive 1116a, flash drive memory 1134, among other types of memory storage. External memory storage device 1124 is connectable to the computer device via USB cable 1156. Each of the memory storage devices, or the memory storage media (1106, 1116, 1118, 1120, 1124, 1134, 1136, and 1138, among others), can contain parts or components, or in its entirety, executable software programming code or application that has been float reduction App 1004/1010 according to aspects of the embodiments, which can implement part or all of the portions of methods and processes shown in FIGS. 1-9, among other methods/processes not shown, described herein.

In addition to the above described components, the computer device also comprises keyboard 1128, external display 1126, printer/scanner/fax machine 1160, and mouse 1130 (although not technically part of the computer device, the peripheral components as shown in FIGS. 11 (1124, 1126, 1128, 1130, 1134, 1136, 1138, 1156, 1158, 1160, and 1162) are well known and adapted for use with the computer device that for purposes of this discussion they shall be considered as being part of the computer device). Other cable types that can be used with the computer device include RS 232, among others, not shown, that can be used for one or more of the connections between the computer device and the peripheral components described herein. Keyboard 1128, and mouse 1130 are connectable to the computer device via USB cable 56, and external display 1126 is connectible to the computer device via VGA cable/HDMI cable 1162. The computer device is connectible to network 1006 via Ethernet port 1111 and Ethernet cable 1158 via a router and modulator-demodulator (MODEM) and internet service provider, none of which are shown in FIG. 11. All of the immediately aforementioned components (1122, 1124, 1126, 1128, 1130, 1134, 1136, 1138, 1156, 1158, and 1160) are known to those of ordinary skill in the art, and this description includes all known and future variants of these types of devices.

External display 1126 can be any type of known display or presentation screen, such as liquid crystal displays (LCDs), light emitting diode displays (LEDs), plasma displays, cathode ray tubes (CRTs), among others (including touch screen displays). In addition to the user interface mechanism such as mouse 1130, the computer device can further include a microphone, touch pad, joy stick, touch screen, voice-recognition system, among other inter-active inter-communicative devices/programs, which can be used to enter data and voice, and which all of are known to those of skill in the art and thus a detailed discussion thereof has been omitted in fulfillment of the dual purposes of clarity and brevity.

As mentioned above, the computer device further comprises a plurality of wireless transceiver devices, such as Wi-Fi transceiver 1142, BT transceiver 1144, NFC transceiver 1146, cellular transceiver 1148, satellite transceiver 1150, and antenna 1152. While each of Wi-Fi transceiver 1142, BT transceiver 1144, NFC transceiver 1146, cellular transceiver 1148, and satellite transceiver 1150 has their own specialized functions, each can also be used for other types of communications, such as accessing a cellular service provider (not shown), accessing network 1006 (which can include the Internet), texting, emailing, among other types of communications and data/voice transfers/exchanges, as known to those of skill in the art. Each of Wi-Fi transceiver 1142, BT transceiver 1144, NFC transceiver 1146, cellular transceiver 1148, satellite transceiver 1150 includes a transmitting and receiving device, and a specialized antenna, although in some instances, one antenna can be shared by one or more of Wi-Fi transceiver 1142, BT transceiver 1144, NFC transceiver 1146, cellular transceiver 1148, and satellite transceiver 1150. Alternatively, one or more of Wi-Fi transceiver 1142, BT transceiver 1144, NFC transceiver 1146, cellular transceiver 1148, and satellite transceiver 1150 will have a specialized antenna, such as satellite transceiver 1150 to which is electrically connected at least one antenna 1152.

In addition, the computer device can access network 1006 (which can be the Internet), either through a hard wired connection such as Ethernet port 1111 as described above, or wirelessly via Wi-Fi transceiver 1142, cellular transceiver 1148 and/or satellite transceiver 1150 (and their respective antennas) according to aspects of the embodiments. The computer device can also be part of a larger network configuration as in a global area network (GAN) (e.g., internet), which ultimately allows connection to various landlines.

According to further aspects of the embodiments, integrated touch screen display 1102, keyboard 1128, mouse 1130, and external display 1126 (if in the form of a touch screen), can provide a means for a user to enter commands, data, digital, and analog information into the computer device. Integrated and external displays 1102, 1126 can be used to show visual representations of acquired data, and the status of applications that can be running, among other things.

Bus 1104 provides a data/command pathway for items such as: the transfer and storage of data/commands between processor 1108, Wi-Fi transceiver 1142, BT transceiver 1144, NFC transceiver 1146, cellular transceiver 1148, satellite transceiver 1150, integrated display 1102, USB port 1110, Ethernet port 1111, VGA/HDMI port 1122, CD/DVD/RW drive 1112, floppy diskette drive 1114, and internal memory 1132. Through bus 1104, data can be accessed that is stored in internal memory 1132. Processor 1108 can send information for visual display to either or both of integrated and external displays 1102, 1126, and the user can send commands to the system via float reduction App 1004/1010 that might reside in processor internal memory 1106 of processor 1108, or any of the other memory devices (1136, 1138, 1116, 1118, and 1120).

The computer device, and either processor internal memory 1106 or internal memory 1132, can be used to implement one or more, or any combination thereof, methods 1400, 1500, and 1600, as well as those not shown and discussed, for creating essays using a computer device according to aspects of the embodiments. Hardware, firmware, software, or a combination thereof can be used to perform the various steps and operations described herein. According to aspects of the embodiments, float reduction App 1004/1010 for carrying out the above discussed steps can be stored and distributed on multi-media storage devices such as devices 1116, 1118, 1120, 1134, 1136 and/or 1138 (described above) or other form of media capable of portably storing information. Storage media 1134, 1136 and/or 1138 can be inserted into, and read by devices such as USB port 1110, CD/DVD/RW drive 1112, and floppy disk drive 1114, respectively.

As also will be appreciated by one skilled in the art, the various functional aspects of the aspects of the embodiments can be embodied in a wireless communication device, a telecommunication network, or as a method or in a computer program product. Accordingly, aspects of embodiments can take the form of an entirely hardware embodiment or an embodiment combining hardware and software aspects. Further, the aspects of embodiments can take the form of a computer program product stored on a computer-readable storage medium having computer-readable instructions embodied in the medium. Any suitable computer-readable medium can be utilized, including hard disks, CD-ROMs, DVDs, optical storage devices, or magnetic storage devices such a floppy disk or magnetic tape. Other non-limiting examples of computer-readable media include flash-type memories or other known types of memories.

Further, those of ordinary skill in the art in the field of the aspects of the embodiments can appreciate that such functionality can be designed into various types of circuitry, including, but not limited to field programmable gate array structures (FPGAs), application specific integrated circuitry (ASICs), microprocessor based systems, among other types. A detailed discussion of the various types of physical circuit implementations does not substantively aid in an understanding of the aspects of the embodiments, and as such has been omitted for the dual purposes of brevity and clarity. However, as well known to those of ordinary skill in the art, the systems and methods discussed herein can be implemented as discussed and can further include programmable devices.

Such programmable devices and/or other types of circuitry as previously discussed can include a processing unit, a system memory, and a system bus that couples various system components including the system memory to the processing unit. The system bus can be any of several types of bus structures including a memory bus or memory controller, a peripheral bus, and a local bus using any of a variety of bus architectures. Furthermore, various types of computer readable media can be used to store programmable instructions. Computer readable media can be any available media that can be accessed by the processing unit. By way of example, and not limitation, computer readable media can comprise computer storage media and communication media. Computer storage media includes volatile and nonvolatile as well as removable and non-removable media implemented in any method or technology for storage of information such as computer readable instructions, data structures, program modules or other data. Computer storage media includes, but is not limited to, RAM, ROM, EEPROM, flash memory or other memory technology, CD-ROMs, DVDs or other optical disk storage, magnetic cassettes, magnetic tape, magnetic disk storage or other magnetic storage devices, or any other medium which can be used to store the desired information and which can be accessed by the processing unit. Communication media can embody computer readable instructions, data structures, program modules or other data in a modulated data signal such as a carrier wave or other transport mechanism and can include any suitable information delivery media.

The system memory can include computer storage media in the form of volatile and/or nonvolatile memory such as ROM and/or RAM. A basic input/output system (BIOS), containing the basic routines that help to transfer information between elements connected to and between the processor, such as during start-up, can be stored in memory. The memory can also contain data and/or program modules that are immediately accessible to and/or presently being operated on by the processing unit. By way of non-limiting example, the memory can also include an operating system, application programs, other program modules, and program data.

The processor can also include other removable/non-removable and volatile/nonvolatile computer storage media. For example, the processor can access a hard disk drive that reads from or writes to non-removable, nonvolatile magnetic media, a magnetic disk drive that reads from or writes to a removable, nonvolatile magnetic disk, and/or an optical disk drive that reads from or writes to a removable, nonvolatile optical disk, such as a CD-ROM or other optical media. Other removable/non-removable, volatile/nonvolatile computer storage media that can be used in the operating environment include, but are not limited to, magnetic tape cassettes, flash memory cards, digital versatile disks, digital video tape, solid state RAM, solid state ROM and the like. A hard disk drive can be connected to the system bus through a non-removable memory interface such as an interface, and a magnetic disk drive or optical disk drive can be connected to the system bus by a removable memory interface, such as an interface.

Aspects of the embodiments discussed herein can also be embodied as computer-readable codes on a computer-readable medium. The computer-readable medium can include a computer-readable recording medium and a computer-readable transmission medium. The computer-readable recording medium is any data storage device that can store data which can be thereafter read by a computer system. Examples of the computer-readable recording medium include ROM, RAM, CD-ROMs and generally optical data storage devices, magnetic tapes, flash drives, and floppy disks. The computer-readable recording medium can also be distributed over network coupled computer systems so that the computer-readable code is stored and executed in a distributed fashion. The computer-readable transmission medium can transmit carrier waves or signals (e.g., wired, or wireless data transmission through the Internet). Also, functional programs, codes, and code segments to, when implemented in suitable electronic hardware, accomplish or support exercising certain elements of the appended claims can be readily construed by programmers skilled in the art to which the aspects of the embodiments pertains.

The disclosed aspects of the embodiments provide a system and method for reducing float for healthcare providers using one or more computer devices. It should be understood that this description is not intended to limit aspects of the embodiments. On the contrary, aspects of the embodiments are intended to cover alternatives, modifications, and equivalents, which are included in the spirit and scope of the aspects of the embodiments as defined by the appended claims. Further, in the detailed description of the aspects of the embodiments, numerous specific details are set forth to provide a comprehensive understanding of the claimed aspects of the embodiments. However, one skilled in the art would understand that various aspects of the embodiments can be practiced without such specific details.

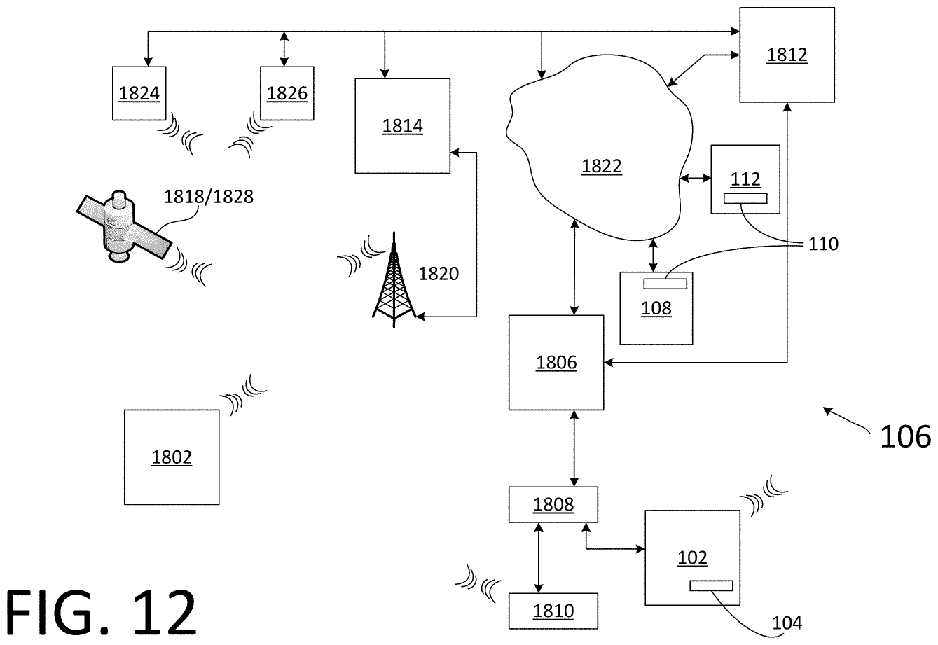

FIG. 12 illustrates network system 1006 within which the system and method for reducing float for a healthcare provider on one or more computer devices can be implemented according to aspects of the embodiments. Much of the infrastructure of network system 1006 shown in FIG. 12 is or should be known to those of skill in the art, so, in fulfillment of the dual purposes of clarity and brevity, a detailed discussion thereof shall be omitted.

According to aspects of the embodiments, a user of the above described system and method can store FRT App 1004 on their mobile device 1202, as well as on PED 1002. Mobile devices 1202 can include, but are not limited to, so-called smart phones, tablets, personal digital assistants (PDAs), notebook and laptop computers, and essentially any device that can access the internet and/or cellular phone service or can facilitate transfer of the same type of data in either a wired or wireless manner.

Both mobile device 1202 and PED 1002 can access cellular service provider 1214, either through a wireless connection (cellular tower 1220) or via a wireless/wired interconnection (a “Wi-Fi” system that comprises, e.g., modulator/demodulator (modem) 1208, wireless router 1210, internet service provider (ISP) 1206, and internet 1222). Further, mobile device 1202 and PED 1002 can include NFC, “Wi-Fi,” and Bluetooth (BT) communications capabilities as well, all of which are known to those of skill in the art. To that end, network system 1006 further includes, as many businesses (and homes do), one or more PEDs 1002 that can be connected to wireless router 1210 via a wired connection (e.g., modem 1208) or via a wireless connection (e.g., Bluetooth). Modem 1208 can be connected to ISP 1206 to provide internet-based communications in the appropriate format to end users (e.g., PED 1002), and which takes signals from the end users and forwards them to ISP 1206. Such communication pathways are well known and understand by those of skill in the art, and a further detailed discussion thereof is therefore unnecessary.

Mobile device 1202 and PEDs 1002 can also access global positioning system (GPS) satellite 1228, which is controlled by GPS station 1224, to obtain positioning information (which can be useful for different aspects of the embodiments), or mobile device 1202 and PEDs 1002 can obtain positioning information via cellular service provider 1214 using cellular 1222 tower(s) 1220 according to one or more well-known methods of position determination. Some mobile devices 1202 can also access communication satellites 1218 and their respective satellite communication systems control stations 1226 (the satellite in FIG. 12 is shown common to both communications and GPS functions) for near-universal communications capabilities, albeit at a much higher cost than convention “terrestrial” cellular services. Mobile device 1202 can also obtain positioning information when near or internal to a building (or arena/stadium) through the use of one or more of NFC/BT devices, the details of which are known to those of skill in the art. FIG. 12 also illustrates other components of network 1006 such as plain old telephone service (POTS) provider 1212.

According to further aspects of the embodiments, and as described above, network 1006 also contains servers 1008, 1012 that can include FRM App 1010, wherein one or more processors, using known and understood technology, such as memory, data and instruction buses, and other electronic devices, can store and implement code that can implement the system and method for creating essays on a computer device according to aspects of the embodiments.

As described above, an encoding process is discussed specifically in reference to FIGS. 1-9, although such delineation is not meant to be, and should not be taken in a limiting manner, as additional methods according to aspects of the embodiments have been described herein. The encoding processes as described are not meant to limit the aspects of the embodiments, or to suggest that the aspects of the embodiments should be implemented following the encoding processes. The purpose of the encoding processes as described is to facilitate the understanding of one or more aspects of the embodiments and to provide the reader with one or many possible implementations of the processed discussed herein. FIGS. 1-9 illustrates flowcharts and/or process steps of various method steps performed during the encoding process, but such encoding processes are not limited thereto. The steps of FIGS. 1-9 are not intended to completely describe the encoding processes but only to illustrate some of the aspects discussed above.

### Additional Embodiments

Other objects of the invention are achieved by providing computerized methods and system for reducing task complexity of patient practice management.

Other objects of the present invention are achieved by providing a computerized management method for reducing task complexity of patient practice management, the method comprising: software executing on a processor configured to:

communicate with at least one server,
access at least one database to compile a case status list,
generate an action list of tasks that may be performed on said cases, expand said action list to further include all candidates capable of performing said tasks,
prepare said expanded action list for evaluation, wherein unlikely candidates are removed while candidates with a substantial probability of having high potential value are retained,
calculate potential value of said tasks and candidates,
select the highest potential value task and candidate pair, and delegate said selected task to said selected candidate.

In certain embodiments, the potential value is calculated based on minimizing Payor float.

In certain embodiments, the delegating said selected task comprises placing said selected task on said selected candidate's workbench.

In certain embodiments, the method includes transmitting said task to the workbench of the selected candidate that is responsible for that function.

It In certain embodiments, the delegating said task further comprises providing all relevant information.

In certain embodiments, the delegating said task further comprises providing only relevant information.

In certain embodiments, the method includes comprising reducing errors and delays.

In certain embodiments, the said at least one database is selected from a group consisting of patients, providers, practice management staff, claims, claim validation rules, tasks, process description, or a combination thereof.

Other objects of the invention are achieved by providing a computerized management system useful in delegating tasks, the system comprising:

a processor for accessing at least one database on a server to compile an action list and delegate tasks;
a custom software/algorithm based on game theory used to evaluate potential value of each business action; and
a workbench for receiving delegated tasks based on high potential value.

In certain embodiments, the processor is configured to:

communicate with at least one server,
access at least one database to compile a case status list,
generate an action list of tasks that may be performed on said cases,
expand said action list to further include all candidates capable of performing said tasks,
prepare said expanded action list for evaluation, wherein unlikely candidates are removed while candidates with a substantial probability of having high potential value are retained,
calculate potential value of said tasks and candidates,
select the highest potential value task and candidate pair, and
delegate said selected task to said selected candidate.

In certain embodiments, the system continuously iterates through available databases and selects the best action for a workbench.

It In certain embodiments, the system is in communication with the Payor in order to send claims, appeals, notifications and receive notifications and payments.

In certain embodiments, potential value is calculated based on minimizing Payor float.

In certain embodiments, potential value is calculated based on minimizing payment delay.

It In certain embodiments, the action list is assembled by acquiring a pending case list, generating tasks for each case, and cross referencing said tasks with capable candidates.

It In certain embodiments, the action list is pruned to remove unlikely candidates identified and selected based on low potential value.

In certain embodiments, the processor compiles a list of all possible actions that may be executed per case.

In certain embodiments, the processor expands the list of possible actions to include alternative candidates for executing said action.

### Embodiments Non-Limiting

What is contemplated by the instant invention is the novel and useful transforming of practice management and Payor interaction into a theoretical-game framework; and applying those concepts in the practice management context.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

The disclosed embodiments provide a source array, computer software, and a method for reducing Payor float. It should be understood that this description is not intended to limit the embodiments. On the contrary, the embodiments are intended to cover alternatives, modifications, and equivalents, which are included in the spirit and scope of the embodiments as defined by the appended claims. Further, in the detailed description of the embodiments, numerous specific details are set forth to provide a comprehensive understanding of the claimed embodiments. However, one skilled in the art would understand that various embodiments can be practiced without such specific details.

Although the features and elements of aspects of the embodiments are described being in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and can include other examples that occur to those skilled in the art. Such other examples are intended to be within the scope of the claims.

The above-described embodiments are intended to be illustrative in all respects, rather than restrictive, of the embodiments. Thus, the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

1. A float reduction system comprising:
at least one processor communicatively couple to at least one database server;
a memory operatively connected to the at least one processor, wherein the memory stores computer executable instructions that, when executed by the at least one processor, causes the at least one processor to execute a method that comprises:
accessing the at least one database that includes a plurality of patient claim data items for a healthcare service provider (payee);
determining a weighting factor for each of a plurality of patient claim data items for each of a plurality of healthcare insurance providers (payors) to determine a potential float reduction (PFR), wherein the weighting factor takes into account respective payor difficulty in making payments to the payee and heuristic learning of previous attempts to collect payments from each respective payor to the payee, wherein the determined weighting factor is substantially continuously determined;
applying the determined weighting factor to each of a plurality of patient claim data items for respective payors to generate a normalized list of patient claim data items;
generating a list of action items based upon the normalized list of patient claim data items;
sorting the list of action items based on the normalized patient claim data, such that normalized patient claim data is sorted by PFR from highest to lowest; and
performing further processing of claims based on the sorted list of action items such that those with the highest PFR are processed soonest, thereby reducing float owed to the payee.

2. The float reduction system according to claim 1, wherein method that is executed by the at least one processor further comprises:
adjusting the weighting factor based upon batch processing such that patient claim data items are sorted into groups and combinations of groups contribute to payor float.

3. The float reduction system according to claim 1, wherein method that is executed by the at least one processor further comprises:
configuring the weighting factor based upon specific rules, the rules being followed to create the ranked list of patient claim data.

4. The float reduction system according to claim 1, wherein method that is executed by the at least one processor further comprises:
adjusting the weighting factor based upon current procedural terminology (CPT) codes and in-network reimbursement fees.

5. The float reduction system according to claim 1, wherein the method that is executed by the at least one processor further comprises:
providing a workflow of action items for each day.

6. The float reduction system according to claim 5, wherein the workflow of action items for each day substantially minimizes overall payor float.

7. The float reduction system according to claim 1, wherein the method that is executed by the at least one processor further comprises:
providing a list of action items to work on for each day; and
delegating them to individual staff at a healthcare institution.

8. The float reduction system according to claim 1, wherein the method that is executed by the at least one processor further comprises:
generating a task list on a work bench for multiple users.

9. The float reduction system according to claim 1, wherein said at least one database is selected from a group consisting of patients, providers, practice management staff, claims, claim validation rules, tasks, process description, or a combination thereof.

10. The float reduction system according to claim 1, wherein the method that is executed by the at least one processor further comprises:
communicating via electronic communication systems with the payor in order to send claims, appeals, notifications and receive notifications and payments.

11. A method for minimizing overall payor float in a health insurance billing system, the method comprising:
accessing at least one database that includes a plurality of patient claim data items for a healthcare service provider (payee), wherein
the at least one database is stored in a memory operatively connected to at least one processor, wherein the memory stores computer executable instructions that, when executed by the at least one processor, causes the at least one processor to execute the method, and wherein the at least one database and at least processor comprise the health insurance billing system (system);
determining a weighting factor for each of a plurality of patient claim data items for each of a plurality of healthcare insurance providers (payors) to determine a potential float reduction (PFR), wherein the weighting factor takes into account respective payor difficulty in making payments to the payee and heuristic learning of previous attempts to collect payments from each respective payor to the payee, wherein the determined weighting factor is substantially continuously determined;
applying the determined weighting factor to each of a plurality of patient claim data items for respective payors to generate a normalized list of patient claim data items;
generating a list of action items based upon the normalized list of patient claim data items;
sorting the list of action items based on the normalized patient claim data, such that normalized patient claim data is sorted by PFR from highest to lowest; and
performing further processing of claims based on the sorted list of action items such that those with the highest PFR are processed soonest, thereby reducing float owed to the payee.

12. The method according to claim 11, further comprising:
adjusting the weighting factor based upon batch processing such that patient claim data items are sorted into groups and combinations of groups contribute to payor float.

13. The method according to claim 11, further comprising:
configuring the weighting factor based upon specific rules, the rules being followed to create the ranked list of patient claim data.

14. The method according to claim 11, wherein further comprising:
adjusting the weighting factor based upon current procedural terminology (CPT) codes and in-network reimbursement fees.

15. The method according to claim 11, wherein further comprising:
providing a workflow of action items for each day.

16. The method according to claim 15, wherein
the workflow of action items for each day minimizes overall payor float.

17. The method according to claim 11, further comprising:
providing a list of action items to work on for each day; and
delegating the list of action items to individual staff at a healthcare institution.

18. The method according to claim 11, further comprising:
generating a task list on a work bench for multiple users.

19. The method according to claim 11, wherein
the at least one database is selected from a group consisting of patients, providers, practice management staff, claims, claim validation rules, tasks, process description, or a combination thereof.

20. The method according to claim 11, further comprising:
communicating with the payor via an electronic communications system to send claims, appeals, notifications and receive notifications and payments.

21. A non-transitory computer readable storage medium storing a computer program product for minimizing payor float in a healthcare insurance billing system, the non-transitory computer readable storage medium comprising computer executable instructions and data, the computer executable instructions able to execute a computer program able to:
determine a weighting factor for each of a plurality of patient claim data items for each of a plurality of healthcare insurance providers (payors) to determine a potential float reduction (PFR), wherein the weighting factor takes into account respective payor difficulty in making payments to the payee and heuristic learning of previous attempts to collect payments from each respective payor to the payee, wherein the determined weighting factor is substantially continuously determined;
apply the determined weighting factor to each of a plurality of patient claim data items for respective payors to generate a normalized list of patient claim data items;
generate a list of action items based upon the normalized list of patient claim data items;
sort the list of action items based on the normalized patient claim data items, such that normalized patient claim data is sorted by PFR from highest to lowest; and
perform further processing of claims based on the sorted list of action items such that those with the highest PFR are processed soonest, thereby reducing float owed to the payee.
